(19) United States
(12) Patent Application Publication
(10) Pub. No.: US 2026/0295094 A1
(43) Pub. Date: Oct. 1, 2026

(54) ENHANCED ELCTROPORATION METHODS FOR LYMPH NODE GENE ELECTROTRANSFER AND CARGO DELIVERY FOR IN SITU T-CELL ENGINEERING

(71) Applicant: Georgia Tech Research Corporation, Atlanta, GA (US)

(72) Inventors: Edward Jacobs, Atlanta, GA (US); Rafael Davalos, Atlanta, GA (US); Julio Arroyo, Atlanta, GA (US); Pedro Santos, Atlanta, GA (US)

(21) Appl. No.: 19/633,472

(22) Filed: Mar. 30, 2026

Related U.S. Application Data
(60) Provisional application No. 63/779,791, filed on Mar. 28, 2025.

(51) Int. Cl. A61K 48/00

(52) U.S. Cl. CPC A61K 48/0075

(57) ABSTRACT

The present disclosure relates generally to electroporation systems and methods for delivering pulsed electric fields to biological tissue, and more particularly to constant-power electroporation techniques for achieving consistent gene electrotransfer outcomes in tissues with variable electrical conductivity. A method for inducing electroporation in biological tissue includes positioning at least one electrode in proximity to the biological tissue, delivering a pulsed electric field to the biological tissue through the at least one electrode, monitoring a voltage and a current delivered to the biological tissue during delivery of the pulsed electric field, and adjusting at least one of the voltage or the current to maintain a substantially constant power delivered to the biological tissue, wherein the substantially constant power produces a substantially constant power density distribution within the biological tissue that is independent of electrical conductivity variations in the biological tissue.

### CROSS-REFERENCE TO RELATED APPLICATIONS

This application claims the benefit of U.S. Provisional Application Serial No. 63/779,791, filed on 28 Mar. 2025, which is incorporated herein by reference in its entirety as if fully set forth below.

### GOVERNMENT LICENSE RIGHTS

This invention was made with government support under CA240476 awarded by the National Institutes of Health. The government has certain rights in the invention.

### FIELD OF INVENTION

The present disclosure relates to electroporation systems and methods for delivering pulsed electric fields to biological tissue, and more particularly to constant-power electroporation techniques for achieving consistent gene electrotransfer outcomes in tissues with variable electrical conductivity, including in situ transfection of T-cells within lymph nodes for generating autologous cell therapies.

### BACKGROUND

The field of gene therapy and cellular engineering has advanced considerably, with various technologies being developed to deliver genetic material into cells for therapeutic purposes. Current gene delivery methods include viral vectors, lipid nanoparticles, and electroporation systems. Viral vectors utilize modified viruses to transport genetic cargo into target cells, while lipid nanoparticles encapsulate nucleic acids for delivery across cell membranes. Electroporation systems apply pulsed electric fields to biological tissue to temporarily increase cell membrane permeability, thereby facilitating the uptake of genetic material such as DNA, mRNA, proteins, and CRISPR components. These existing technologies have been applied across diverse therapeutic areas, including cancer immunotherapy, treatment of genetic disorders, and regenerative medicine. In the context of adoptive cell therapies, such as chimeric antigen receptor T-cell (CAR-T) therapies, current manufacturing processes typically involve ex vivo isolation of T-cells from patients, activation and genetic modification of the cells in laboratory settings, expansion of the modified cells, and subsequent reinfusion into patients.

However, existing gene delivery technologies suffer from several limitations. Viral vectors face challenges related to immunogenicity, potential mutagenicity from genomic integration, and constraints on cargo size. Lipid nanoparticles exhibit inconsistent biodistribution to target tissues such as lymph nodes and variable delivery efficiency into cells, with populations of the same cell type often having variable expression of target markers that nanoparticles rely upon for enhanced transport. Current electroporation systems typically operate by delivering constant voltage pulses to tissue, but the electric field threshold that induces reversible electroporation is negatively correlated with tissue electrical conductivity. This dependency on tissue electrical properties creates variability in electroporation outcomes, as both the electric field distribution and the electroporation threshold are affected by tissue conductivity, which varies between patients, between healthy and diseased tissues, and changes non-linearly during the electroporation process itself due to heating and pore formation. Consequently, protocols optimized in controlled ex vivo environments with known electrical conductivity do not translate reliably to in vivo applications where tissue properties are uncertain and cannot be easily controlled. Additionally, current ex vivo CAR-T manufacturing pipelines are time-consuming, typically requiring several weeks to months, and expensive, with treatment costs ranging from $350,000 to $500,000 per patient. The multiple steps involved in ex vivo manufacturing, including cell isolation, transport, activation, genetic modification, sorting, expansion, purification, and reinfusion, can diminish T-cell efficacy through premature exhaustion and accrue costs at each stage.

What is needed, therefore, is an improved electroporation approach that maintains consistent electroporation outcomes regardless of variations in tissue electrical conductivity. Such an approach would enable reliable in vivo gene electrotransfer by controlling the power density delivered to tissue rather than relying on voltage-based parameters that are conductivity-dependent. An improved system would facilitate in situ transfection of cells within target tissues, potentially enabling direct engineering of T-cells within lymph nodes to generate autologous cell therapies without the complexity, cost, and time associated with ex vivo manufacturing processes.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a method for inducing electroporation in biological tissue is provided. The method can include positioning at least one electrode in proximity to the biological tissue. The method can include delivering a pulsed electric field to the biological tissue through the at least one electrode. The method can include monitoring a voltage and a current delivered to the biological tissue during delivery of the pulsed electric field. The method can include adjusting at least one of the voltage or the current to maintain a substantially constant power delivered to the biological tissue, wherein the substantially constant power produces a substantially constant power density distribution within the biological tissue that is independent of electrical conductivity variations in the biological tissue.

According to another aspect of the present disclosure, a system for delivering pulsed electric fields to biological tissue is provided. The system can include at least one electrode configured to be positioned in proximity to the biological tissue. The system can include a pulse generator electrically coupled to the at least one electrode and configured to deliver a pulsed electric field to the biological tissue. The system can include a sensing circuit configured to measure a voltage and a current delivered to the biological tissue. The system can include a controller coupled to the sensing circuit and the pulse generator, the controller configured to adjust at least one of the voltage or the current delivered by the pulse generator to maintain a substantially constant power delivered to the biological tissue, wherein the substantially constant power produces a substantially constant power density distribution within the biological tissue that is independent of electrical conductivity variations in the biological tissue.

According to another aspect of the present disclosure, a method for in situ transfection of T-cells within a lymph node is provided. The method can include injecting a genetic material into the lymph node, wherein the genetic material includes at least one of DNA, mRNA, or a CRISPR component. The method can include positioning at least one electrode in proximity to the lymph node. The method can include delivering a pulsed electric field to the lymph node through the at least one electrode to induce reversible electroporation of T-cells within the lymph node. The method can include controlling a power delivered to the lymph node during delivery of the pulsed electric field to maintain a substantially constant power density within the lymph node, thereby facilitating uptake of the genetic material by the T-cells.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates ex vivo and in vivo approaches for mRNA-based CAR T-cell manufacturing.

FIG. 2 illustrates in vivo anti-tumor CAR T-cell generation through gene electrotransfer of T-cells in a lymph node, according to examples of the disclosed technology.

FIG. 3A illustrates reversible electric field threshold versus conductivity for electroporation, according to examples of the disclosed technology.

FIG. 3B illustrates reversible current density threshold versus conductivity for electroporation, according to examples of the disclosed technology.

FIG. 3C illustrates reversible power density threshold versus conductivity for electroporation, according to examples of the disclosed technology.

FIG. 3D illustrates lethal electric field threshold versus conductivity for electroporation, according to examples of the disclosed technology.

FIG. 3E illustrates lethal current density threshold versus conductivity for electroporation, according to examples of the disclosed technology.

FIG. 3F illustrates lethal power density threshold versus conductivity for electroporation, according to examples of the disclosed technology.

FIG. 4A illustrates simulated electric field distributions and electroporation outcomes under constant voltage conditions, according to examples of the disclosed technology.

FIG. 4B illustrates simulated power density distributions and electroporation outcomes under constant power conditions, according to examples of the disclosed technology.

FIG. 5 illustrates a multi-needle electrode array in a hydrogel tissue mimic, according to examples of the disclosed technology.

FIG. 6A illustrates electroporation areas versus applied power for reversible and irreversible electroporation, according to examples of the disclosed technology.

FIG. 6B illustrates an optimal power range for gene electrotransfer applications, according to examples of the disclosed technology.

FIG. 7 illustrates electroporation zone progression at increasing power levels, according to examples of the disclosed technology.

FIG. 8A illustrates fluid drainage pathways in lymph nodes at zero seconds, according to examples of the disclosed technology.

FIG. 8B illustrates fluid drainage pathways in lymph nodes at thirty seconds, according to examples of the disclosed technology.

FIG. 8C illustrates fluid drainage pathways in lymph nodes at sixty seconds, according to examples of the disclosed technology.

FIG. 8D illustrates fluid drainage pathways in lymph nodes at ninety seconds, according to examples of the disclosed technology.

FIG. 8E illustrates fluid drainage pathways in lymph nodes at one hundred twenty seconds, according to examples of the disclosed technology.

FIG. 9 illustrates fluorescence intensity versus elapsed time for lymph node drainage, according to examples of the disclosed technology.

FIG. 10 illustrates a full-bridge topology circuit for delivering electrical pulses to a biological sample, according to examples of the disclosed technology.

FIG. 11 illustrates a full-bridge topology circuit with a sense resistor for current sensing, according to examples of the disclosed technology.

FIG. 12 illustrates a full-bridge topology circuit with an external transistor for current regulation, according to examples of the disclosed technology.

FIG. 13 is a block diagram of a current feedback loop for constant-current electroporation control, according to examples of the disclosed technology.

FIG. 14 is a block diagram of a constant power control system for electroporation, according to examples of the disclosed technology.

FIG. 15 is a block diagram of a system for delivering pulsed electric fields to biological tissue, according to examples of the disclosed technology.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to electroporation systems and methods for delivering pulsed electric fields to biological tissue. Electroporation is a biophysical phenomenon in which cell membrane permeability increases following application of an external pulsed electric field. Reversible electroporation can be used to deliver therapeutic agents, including chemotherapeutics, genes, CRISPR technologies, and other molecules into cells. However, in vivo applications of electroporation have been limited due to variability in electroporation outcomes caused by differences in tissue electrical conductivity between patients and between different tissue types within the same patient.

Conventional electroporation approaches apply constant voltage pulses to tissue. When constant voltage is applied, the resulting current and power delivered to the tissue change as tissue electrical conductivity changes. The electric field threshold that induces reversible electroporation is negatively correlated with tissue electrical conductivity. As a result, protocols optimized in controlled ex vivo environments are not directly translatable to in vivo systems where tissue conductivity varies and is often unknown prior to treatment. If too high of an electric field is applied due to uncertainty about tissue conductivity, mass transport across the cell membrane can be excessive, inducing irreversible electroporation and cell death rather than the desired reversible electroporation for gene delivery.

The present disclosure provides constant-power electroporation techniques that address these limitations. By maintaining a substantially constant power delivered to biological tissue during pulsed electric field delivery, a substantially constant power density distribution can be achieved within the tissue that is independent of electrical conductivity variations. Power density thresholds for reversible and irreversible electroporation have been found to be independent of tissue electrical conductivity, unlike electric field thresholds or current density thresholds. By controlling power delivery rather than voltage or current alone, consistent reversible electroporation areas and consistent irreversible electroporation areas can be achieved across tissues with different electrical properties.

The disclosed systems and methods can be applied to various therapeutic applications, including gene electrotransfer for delivering DNA, mRNA, proteins, and CRISPR components into target cells. In some cases, the disclosed techniques can enable in situ transfection of T-cells within lymph nodes. By delivering genetic material encoding chimeric antigen receptors to T-cells within lymph nodes and applying constant-power pulsed electric fields to induce reversible electroporation, autologous CAR T-cells can be generated directly within a patient. This in situ approach can reduce the time, cost, and complexity associated with ex vivo CAR T-cell manufacturing processes, which typically involve isolating T-cells from a patient, transporting the cells to a manufacturing facility, activating and expanding the cells, delivering genetic material, sorting and purifying transduced cells, and reinfusing the cells into the patient.

The disclosed constant-power electroporation techniques can compensate for patient-to-patient variability in tissue electrical properties, enabling more predictable and consistent treatment outcomes. The techniques can be applied using various electrode configurations, including needle electrode arrays, catheters, and surface electrodes. The techniques can also be applied with various pulse waveforms, including monophasic pulses, biphasic pulses, and pulses having durations ranging from nanoseconds to milliseconds.

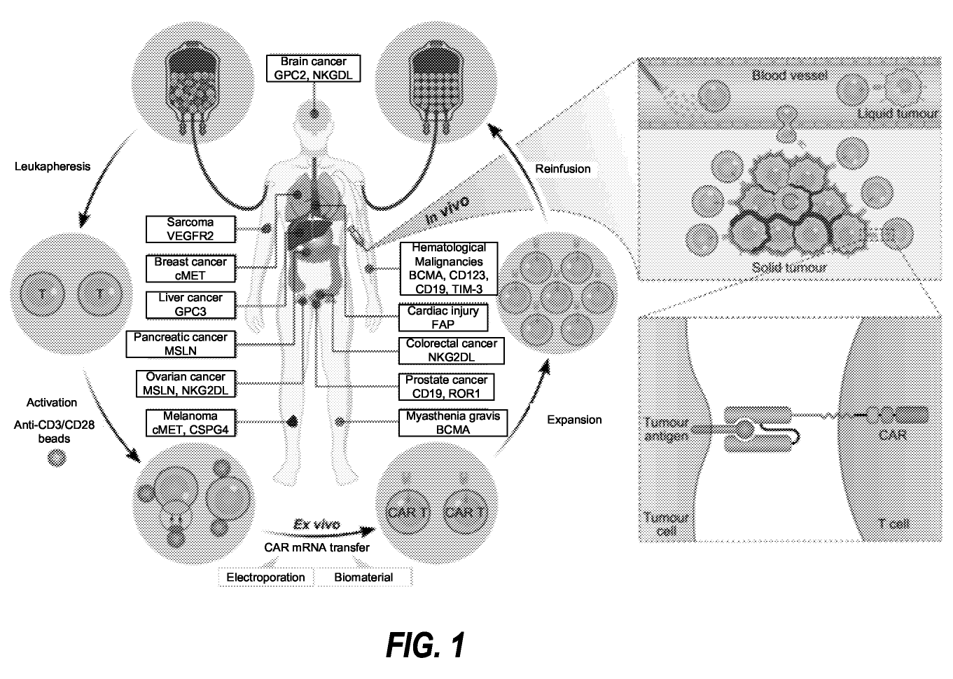

Referring to FIG. 1, ex vivo and in vivo approaches for mRNA-based CAR T-cell manufacturing are illustrated. The ex vivo manufacturing process involves multiple sequential steps. T-cells are first isolated from a patient through leukapheresis, a procedure in which blood is drawn from the patient and T-cells are separated from other blood components. The isolated T-cells, depicted as circular cells, then undergo activation using anti-CD3/CD28 beads, which stimulate the T-cells to proliferate. Following activation, CAR mRNA is transferred into the T-cells through electroporation or biomaterial delivery methods. The cells receiving the CAR mRNA become CAR T-cells expressing chimeric antigen receptors on their surfaces. The CAR T-cells then undergo expansion to increase cell numbers before reinfusion back into the patient.

With continued reference to FIG. 1, the ex vivo manufacturing process can require several weeks to months to complete and can incur costs ranging from $350,000 to $500,000 per treatment. The multiple handling steps involved in ex vivo manufacturing, including transport to manufacturing facilities, enrichment, activation, genetic modification, sorting, expansion, and purification, can diminish T-cell efficacy. Activating and expanding T-cells outside the body can exhaust the cells, with shorter times outside the body correlating with improved responses when the cells are infused back into the patient.

As further shown in FIG. 1, an alternative in vivo approach is illustrated on the right side of the figure. In the in vivo approach, CAR T-cells can be manufactured directly within a patient using targeted mRNA nanocarriers, eliminating the complex procedures associated with ex vivo genetic manipulation. The in vivo approach depicts blood vessels, liquid tumors, and solid tumor environments where CAR T-cells can interact with tumor cells. An inset diagram illustrates the interaction between a tumor cell displaying a tumor antigen and a T-cell expressing a CAR receptor on the T-cell surface.

FIG. 1 also depicts various therapeutic targets organized by tissue location within a human body outline. Brain cancer targets include GPC2 (glypican 2) and NKG2DL (natural killer group 2 member D ligand). Sarcoma targets include VEGFR2 (vascular endothelial growth factor receptor 2). Breast cancer targets include cMET (hepatocyte growth factor receptor). Liver cancer targets include GPC3 (glypican 3). Pancreatic cancer targets include MSLN (mesothelin). Ovarian cancer targets include MSLN and NKG2DL. Melanoma targets include cMET and CSPG4 (chondroitin sulfate proteoglycan 4). Hematological malignancies targets include BCMA (B cell maturation antigen), CD123, CD19, and TIM-3 (T cell immunoglobulin and mucin domain 3). Cardiac injury targets include FAP (fibroblast activation protein). Colorectal cancer targets include NKG2DL. Prostate cancer targets include CD19 and ROR1 (receptor tyrosine kinase-like orphan receptor 1). Myasthenia gravis targets include BCMA. Targets highlighted in red in FIG. 1 indicate those that have been tested in clinical trials.

The in situ CAR T-cell generation approach enabled by the constant-power electroporation techniques disclosed herein can bypass the extensive ex vivo manufacturing pipeline. By engineering T-cells directly within lymph nodes where T-cells reside, the time and cost associated with adoptive cell therapies can be reduced. The in situ approach can also prevent premature T-cell exhaustion that can occur during artificial expansion outside the body.

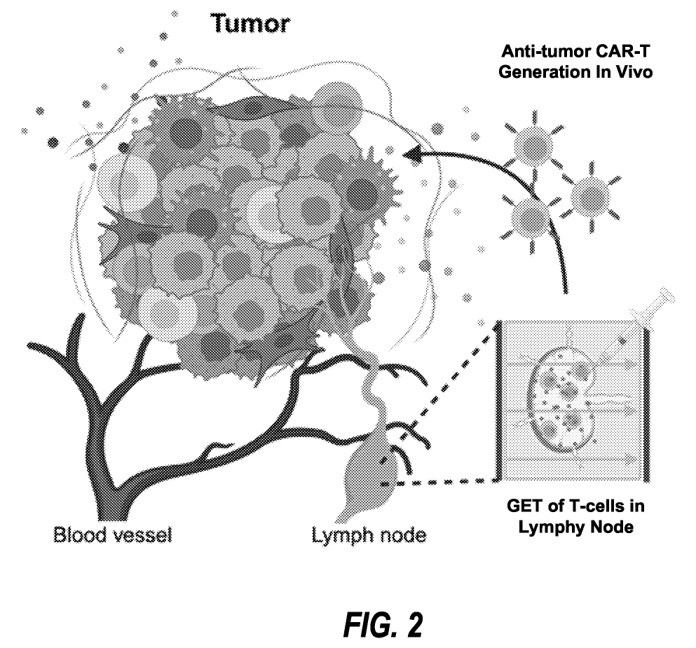

Referring to FIG. 2, a schematic diagram illustrates in vivo anti-tumor CAR T-cell generation through gene electrotransfer of T-cells in a lymph node. The figure depicts a tumor mass containing multiple cells of varying sizes and types, with some cells appearing to have irregular surfaces characteristic of tumor cells. The tumor is connected to underlying vasculature represented by a blood vessel structure and a lymph node. An arrow indicates the migration pathway of anti-tumor CAR T-cells from the lymph node region toward the tumor. The anti-tumor CAR T-cells are depicted with surface projections representing chimeric antigen receptors.

With continued reference to FIG. 2, a detailed inset panel shows the gene electrotransfer process occurring within the lymph node. The method for in situ transfection of T-cells within a lymph node includes injecting a genetic material into the lymph node. The genetic material can comprise at least one of DNA, mRNA, or a CRISPR component. In some cases, the genetic material comprises mRNA encoding a chimeric antigen receptor. A syringe delivers the genetic material into the lymph node tissue, where the genetic material can diffuse throughout the lymph node structure prior to electroporation.

As further shown in FIG. 2, the method includes positioning at least one electrode in proximity to the lymph node. The inset illustrates electrical pulses being applied across the lymph node tissue to facilitate transfection of T-cells. The method includes delivering a pulsed electric field to the lymph node through the at least one electrode to induce reversible electroporation of T-cells within the lymph node. The reversible electroporation increases cell membrane permeability, facilitating uptake of the genetic material by the T-cells residing within the lymph node.

When the genetic material comprises mRNA encoding a chimeric antigen receptor, uptake of the mRNA by the T-cells produces autologous CAR T-cells within the lymph node. The generated CAR T-cells then travel through the lymphatic and circulatory systems to reach and target the tumor cells, as indicated by the migration pathway arrow in FIG. 2.

The method can use the MRL/MpJ-FASlpr mouse model, which exhibits spontaneous lymphadenopathy associated with excessive proliferation of T-cells. In this model, mesenteric lymph nodes grow to 5-8 mm in diameter, resembling human lymph node size of 5-15 mm. The size of the mesenteric lymph nodes in this model allows for optimization of clinically relevant electrode designs and protocols that are not feasible in wild-type mouse lymph nodes, which are approximately 40 times smaller by volume with diameters of 1-2 mm.

The method can include orthotopic injection of CD19+ B-lymphoma cells into the lymph node to create a tumor model for evaluating CAR-T cell generation. After tumors reach a target size, mRNA encoding a CD19 CAR can be injected into a mesenteric lymph node, followed by constant-power gene electrotransfer using optimized protocols. The generated CD19 CAR T-cells can then migrate from the lymph node to target and eliminate the CD19+ B-lymphoma cells.

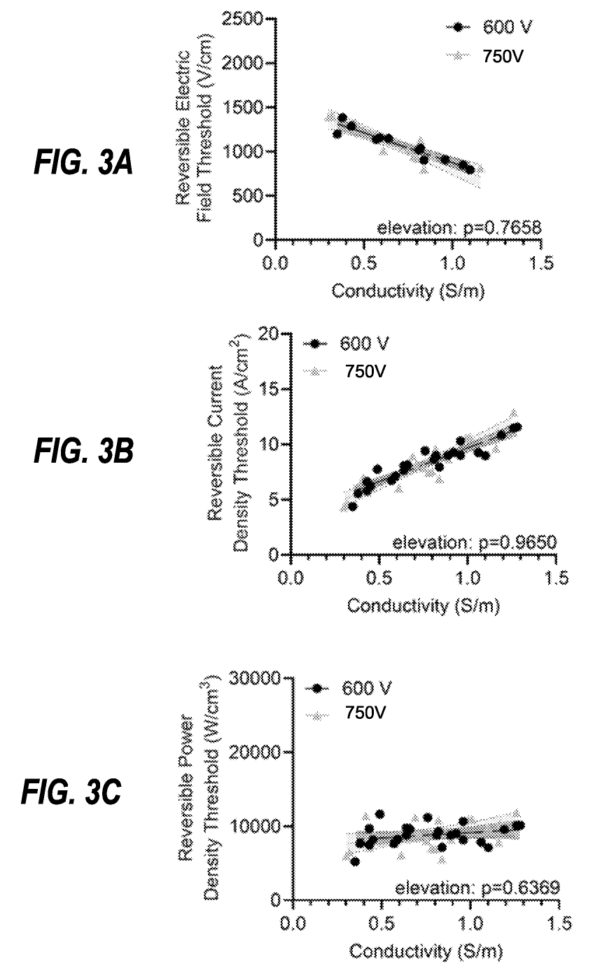

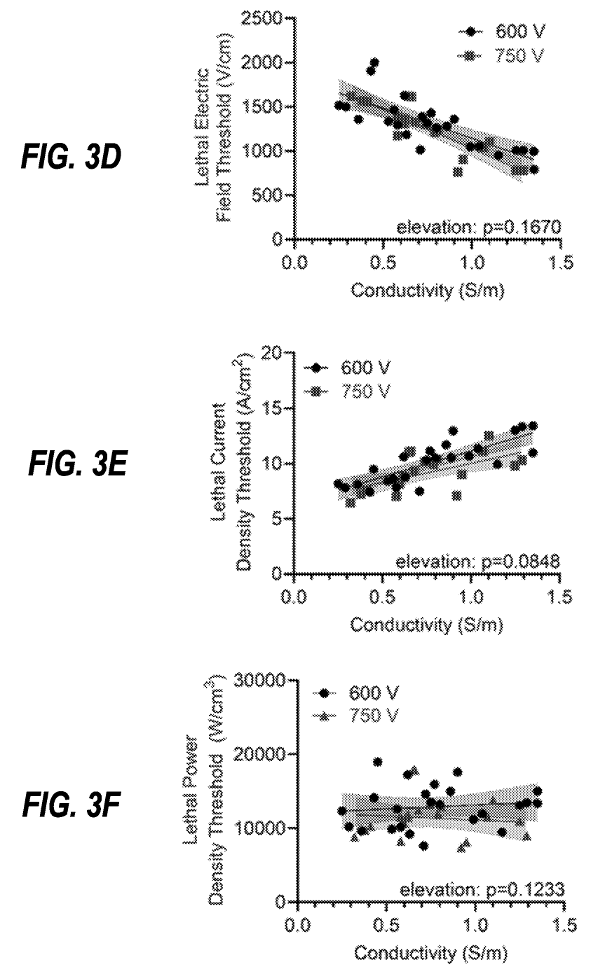

Referring to FIG. 3A, FIG. 3B, and FIG. 3C, experimental data illustrates the relationship between reversible electroporation thresholds and tissue electrical conductivity for two different applied voltages of 600 V and 750 V. The data demonstrates that power density thresholds for reversible electroporation remain substantially constant across conductivity variations, while electric field thresholds and current density thresholds vary with conductivity.

FIG. 3A depicts reversible electric field threshold measured in V/cm on the vertical axis versus conductivity measured in S/m on the horizontal axis. The data points for both voltage conditions show a negative correlation between electric field threshold and conductivity. As conductivity increases from approximately 0.0 S/m to 1.5 S/m, the reversible electric field threshold decreases from approximately 1500 V/cm to approximately 500 V/cm. Dashed lines represent linear regression fits for the respective data sets, with the 600 V data shown as filled circles and the 750 V data shown as triangles. An elevation p-value of 0.7658 indicates no statistically significant difference between the regression lines for the two voltage conditions, confirming that the negative correlation between electric field threshold and conductivity is consistent regardless of the applied voltage magnitude.

With continued reference to FIG. 3B, reversible current density threshold measured in A/cm² is plotted on the vertical axis versus conductivity on the horizontal axis. In contrast to the electric field threshold data, the current density threshold data demonstrates a positive correlation between current density threshold and conductivity. As conductivity increases across the measured range, the reversible current density threshold increases from approximately 5 A/cm² to approximately 15 A/cm². An elevation p-value of 0.9650 indicates no statistically significant difference between the two voltage conditions. The positive correlation between current density threshold and conductivity indicates that applying a constant current density alone does not produce consistent electroporation outcomes across tissues with different electrical properties.

As further shown in FIG. 3C, reversible power density threshold measured in W/cm³ is plotted on the vertical axis versus conductivity on the horizontal axis. The power density threshold values cluster around 5000 to 15000 W/cm³ and remain relatively constant across the conductivity range for both voltage conditions. An elevation p-value of 0.6369 indicates no statistically significant difference between the regression lines. Unlike the electric field threshold data of FIG. 3A and the current density threshold data of FIG. 3B, the power density threshold data of FIG. 3C shows no substantial correlation with conductivity. The regression lines for both voltage conditions are substantially horizontal, indicating that the reversible power density threshold is independent of tissue electrical conductivity.

The data presented in FIG. 3A, FIG. 3B, and FIG. 3C supports the principle that a substantially constant power produces a substantially constant power density distribution within the biological tissue that is independent of electrical conductivity variations in the biological tissue. Because the power density threshold for reversible electroporation does not change with conductivity, maintaining a constant power delivery to tissue results in consistent reversible electroporation outcomes regardless of patient-to-patient variability in tissue electrical properties or variations in electrical properties within different regions of the same tissue.

The substantially constant power density distribution produces substantially consistent reversible electroporation areas within the biological tissue regardless of the electrical conductivity variations. When constant voltage is applied, as shown by the data in FIG. 3A, the electric field threshold changes with conductivity, resulting in variable reversible electroporation areas across tissues with different electrical properties. However, when constant power is applied, the power density distribution remains consistent, and because the power density threshold is independent of conductivity as shown in FIG. 3C, the resulting reversible electroporation areas remain substantially consistent regardless of conductivity variations.

Referring to FIG. 3D, FIG. 3E, and FIG. 3F, experimental data illustrates the relationship between lethal electroporation thresholds and tissue electrical conductivity for two different applied voltages of 600 V and 750 V. The data demonstrates that lethal power density thresholds for irreversible electroporation remain substantially constant across conductivity variations, consistent with the reversible electroporation threshold data presented in FIG. 3A, FIG. 3B, and FIG. 3C.

FIG. 3D depicts lethal electric field threshold measured in V/cm on the vertical axis versus conductivity measured in S/m on the horizontal axis. The data points for both voltage conditions show a negative correlation between lethal electric field threshold and conductivity. As conductivity increases from approximately 0.0 S/m to 1.5 S/m, the lethal electric field threshold decreases from approximately 1500 V/cm to approximately 750 V/cm. Shaded regions represent confidence intervals around the regression lines for each voltage condition. An elevation p-value of 0.1670 indicates no statistically significant difference between the regression lines for the two voltage conditions. The negative correlation between lethal electric field threshold and conductivity parallels the relationship observed for reversible electric field thresholds in FIG. 3A, confirming that electric field thresholds for both reversible and irreversible electroporation are dependent on tissue electrical conductivity.

With continued reference to FIG. 3E, lethal current density threshold measured in A/cm² is plotted on the vertical axis versus conductivity on the horizontal axis. The lethal current density threshold decreases from approximately 10 A/cm² to approximately 7 A/cm² as conductivity increases over the measured range. An elevation p-value of 0.0848 indicates no statistically significant difference between the two voltage conditions. The data demonstrates that lethal current density thresholds, like reversible current density thresholds shown in FIG. 3B, vary with tissue electrical conductivity.

As further shown in FIG. 3F, lethal power density threshold measured in W/cm³is plotted on the vertical axis versus conductivity on the horizontal axis. The lethal power density threshold values remain relatively constant around 10000 W/cm³ across the conductivity range for both voltage conditions. An elevation p-value of 0.1233 indicates no statistically significant difference between the regression lines. The regression lines for both voltage conditions are substantially horizontal, demonstrating that the lethal power density threshold is independent of tissue electrical conductivity.

The data presented in FIG. 3D, FIG. 3E, and FIG. 3F confirms that power density thresholds are independent of tissue electrical conductivity for both reversible and irreversible electroporation. As described previously with respect to FIG. 3C, the substantially constant power density produces a substantially constant power density distribution within the biological tissue that is independent of electrical conductivity variations. The substantially constant power density can be in a range from 1,000 W/cm³ to 10,000 W/cm³. The reversible power density threshold data of FIG. 3C shows values clustering around 5000 to 15000 W/cm³, while the lethal power density threshold data of FIG. 3F shows values around 10000 W/cm³. By selecting a target power density within the range from 1,000 W/cm³ to 10,000 W/cm³, reversible electroporation can be achieved while minimizing irreversible electroporation, thereby facilitating gene electrotransfer applications where cell viability is desired.

The substantially constant power density distribution produces substantially consistent irreversible electroporation areas within the biological tissue regardless of the electrical conductivity variations. When constant voltage is applied, as shown by the data in FIG. 3D, the lethal electric field threshold changes with conductivity, resulting in variable irreversible electroporation areas across tissues with different electrical properties. However, when constant power is applied, the power density distribution remains consistent, and because the lethal power density threshold is independent of conductivity as shown in FIG. 3F, the resulting irreversible electroporation areas remain substantially consistent regardless of conductivity variations.

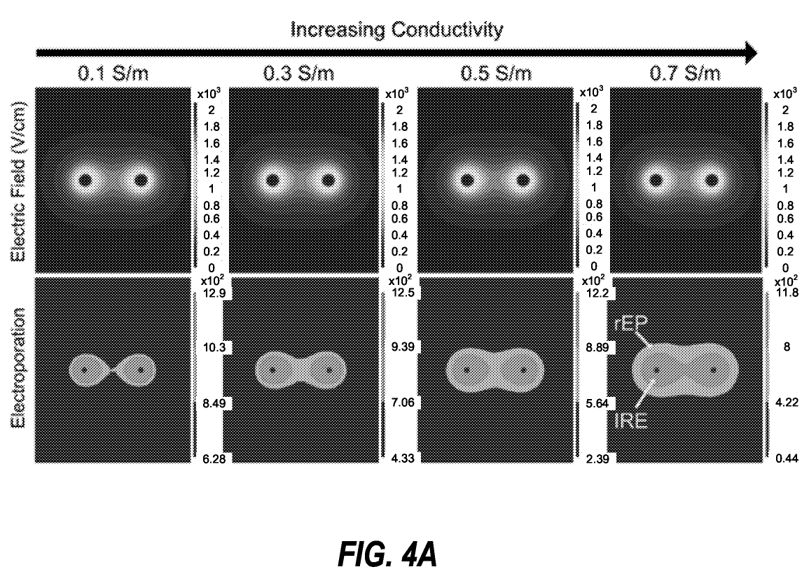

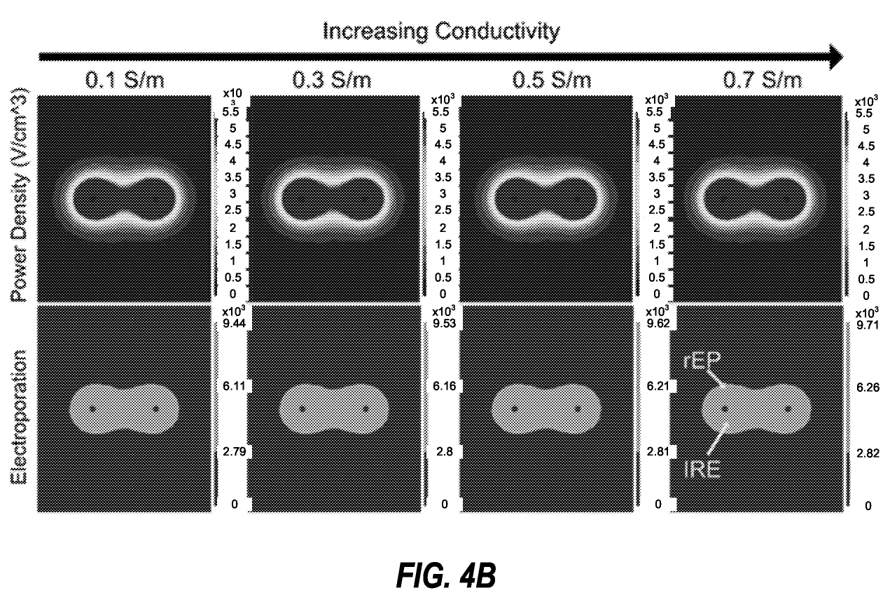

Referring to FIG. 4A, simulated electric field distributions and electroporation outcomes are illustrated for constant voltage conditions across tissues of varying electrical conductivity. The figure is organized as a matrix with two rows and four columns. The top row displays electric field distributions measured in volts per centimeter, while the bottom row displays corresponding electroporation outcomes. The four columns represent increasing conductivity values from left to right, specifically 0.1 S/m, 0.3 S/m, 0.5 S/m, and 0.7 S/m.

In the top row of FIG. 4A, each panel shows a grayscale visualization of the electric field distribution around two electrode positions. A color scale ranges from 0 to 2×10³ V/cm. The electric field pattern remains substantially similar across all conductivity conditions, with high field intensity regions appearing as bright areas surrounding the two electrode locations and lower intensity regions extending outward. When constant voltage is applied, the electric field distribution does not change when varying tissue conductivity because the electric field is determined by the applied voltage and electrode geometry rather than by tissue electrical properties.

With continued reference to FIG. 4A, the bottom row shows the resulting electroporation zones corresponding to the electric field distributions in the top row. The rightmost panel in the bottom row includes labels identifying the reversible electroporation region as rEP and the irreversible electroporation region as IRE. Despite the electric field distribution remaining constant across all conductivity conditions, the electroporation zones change substantially as conductivity increases from left to right. At the lowest conductivity of 0.1 S/m, the reversible electroporation region extends over a larger area relative to the irreversible electroporation region. As conductivity increases to 0.3 S/m, 0.5 S/m, and 0.7 S/m, the irreversible electroporation zone expands relative to the reversible electroporation zone.

The electroporation zone changes observed in FIG. 4A result from the negative correlation between electroporation thresholds and tissue electrical conductivity, as demonstrated by the experimental data in FIG. 3A and FIG. 3D. When constant voltage is applied, the electric field distribution remains fixed, but the electric field threshold that induces reversible or irreversible electroporation decreases as conductivity increases. As a result, a larger portion of the tissue experiences electric field values exceeding the threshold for irreversible electroporation at higher conductivities, causing the irreversible electroporation zone to expand. The reversible electroporation zone, which exists between the reversible and irreversible thresholds, shifts and can narrow as conductivity increases under constant voltage conditions.

Referring to FIG. 4B, simulated power density distributions and electroporation outcomes are illustrated for constant power conditions across tissues of varying electrical conductivity. The figure is organized into two rows and four columns, with conductivity increasing from left to right as indicated by an arrow at the top of the figure. The conductivity values are 0.1 S/m, 0.3 S/m, 0.5 S/m, and 0.7 S/m, matching the conductivity values shown in FIG. 4A.

In the top row of FIG. 4B, each panel shows a cross-sectional view of tissue containing two electrode positions represented as dark circular regions. The power density scale ranges from 0 to approximately 5.5 × 10³ W/cm³ for each conductivity condition. Unlike the electric field distributions shown in FIG. 4A, the power density distributions in FIG. 4B remain substantially consistent across all conductivity conditions. When constant power is applied, the power density distribution within the tissue does not change substantially when varying tissue conductivity.

With continued reference to FIG. 4B, the bottom row displays corresponding electroporation distributions for the same conductivity values. The electroporation panels show regions of reversible electroporation labeled as rEP and regions of irreversible electroporation labeled as IRE. The reversible electroporation regions appear as lighter zones surrounding the electrode positions, while the irreversible electroporation regions appear as darker zones immediately adjacent to the electrodes. In contrast to the electroporation outcomes shown in FIG. 4A under constant voltage conditions, the reversible and irreversible electroporation areas in FIG. 4B remain substantially consistent across different tissue conductivities.

The consistent electroporation outcomes observed in FIG. 4B under constant power conditions result from the independence of power density thresholds from tissue electrical conductivity, as demonstrated by the experimental data in FIG. 3C and FIG. 3F. When constant power is applied, the power density distribution remains substantially constant regardless of conductivity variations. Because the power density thresholds for both reversible and irreversible electroporation are independent of conductivity, the resulting electroporation zones remain substantially consistent across tissues with different electrical properties.

The comparison between FIG. 4A and FIG. 4B illustrates the advantage of constant power electroporation over constant voltage electroporation for achieving consistent treatment outcomes. Under constant voltage conditions as shown in FIG. 4A, the reversible and irreversible electroporation areas change with conductivity, making treatment outcomes unpredictable when tissue electrical properties are unknown or variable. Under constant power conditions as shown in FIG. 4B, the reversible and irreversible electroporation areas remain substantially consistent regardless of conductivity variations, enabling predictable treatment outcomes across patients with different tissue electrical properties and across different tissue types within the same patient.

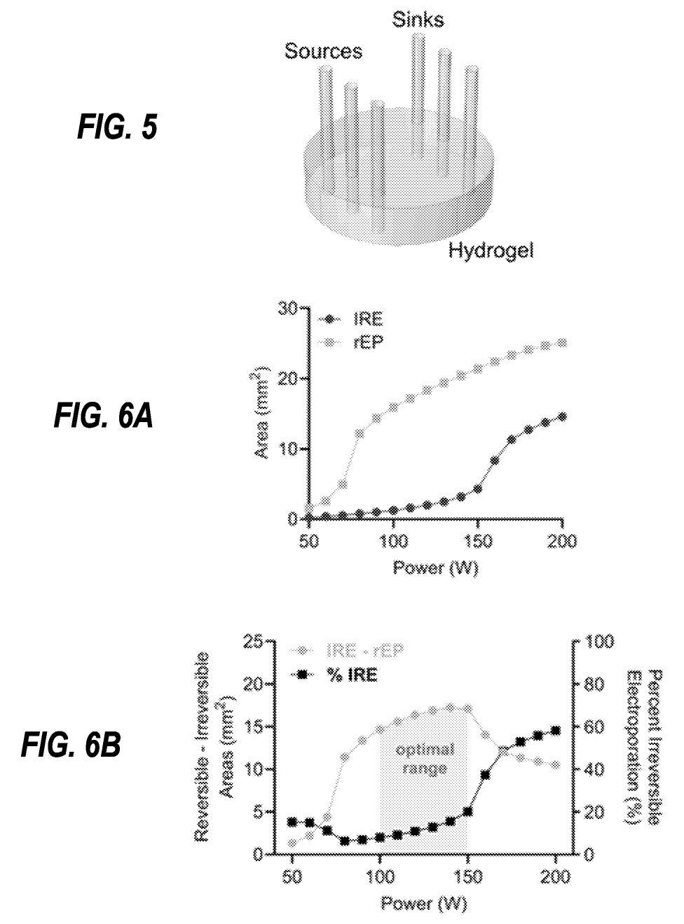

Referring to FIG. 5, a three-dimensional schematic representation illustrates an experimental setup for evaluating constant-power electroporation using a multi-needle electrode array. The setup includes a cylindrical hydrogel with multiple needle electrodes inserted vertically into the hydrogel material. The electrodes are arranged in a circular pattern and are designated as either sources or sinks for electrical current delivery. The at least one electrode can comprise a plurality of needle electrodes arranged in an array configuration. In some cases, the electrode comprises a six-needle electrode array configuration for gene electrotransfer applications.

With continued reference to FIG. 5, the hydrogel serves as a tissue mimic for evaluating electroporation protocols prior to in vivo application. The method can target T-cells embedded within collagen hydrogel at a cellular density of 10⁷T-cells/ml to match lymph node cellular density. The collagen hydrogel can have a concentration of 0.3 mg/ml to match extracellular protein concentration of lymph nodes. Cells embedded within extracellular matrix replicate in vivo morphologies and follow in vivo electroporation trends more closely than cells in suspension. The hydrogel tissue mimic allows for optimization of electrode designs and treatment protocols in a controlled environment before translation to in vivo systems.

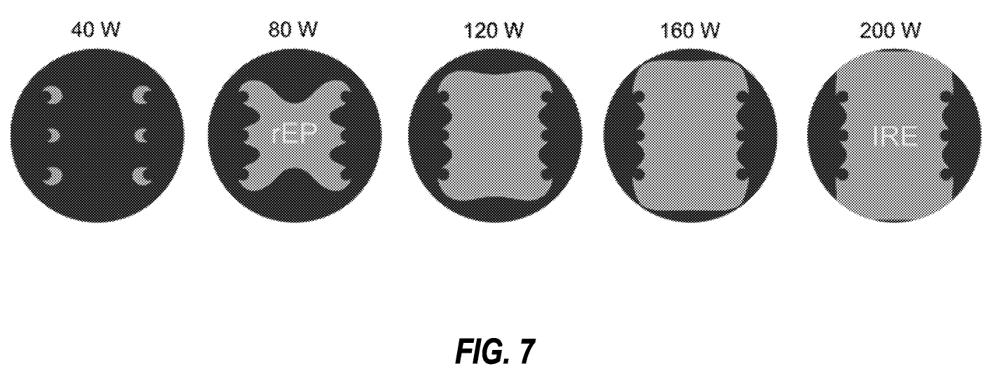

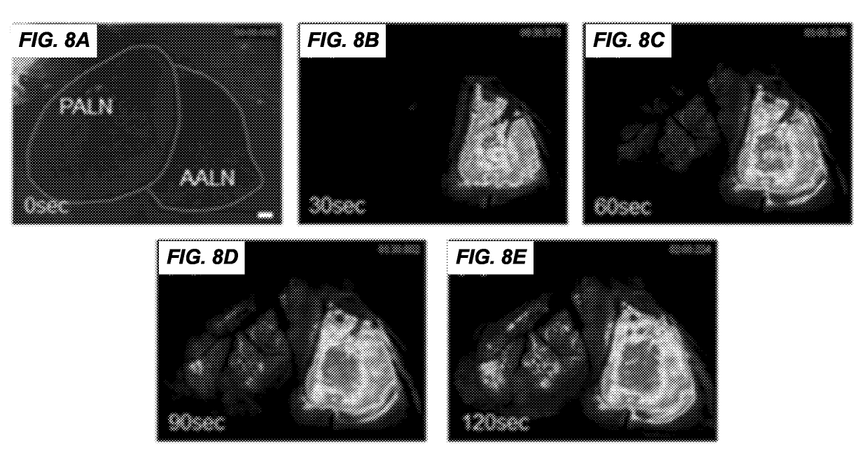

Referring to FIG. 6A, a graph plots area in square millimeters on the vertical axis against power in watts on the horizontal axis. Two curves are shown, one representing irreversible electroporation (IRE) and one representing reversible electroporation (rEP). The reversible electroporation curve demonstrates a steeper increase with increasing power compared to the irreversible electroporation curve. Both curves show minimal electroporation area at approximately 50 watts. As power increases, the reversible electroporation area reaches approximately 25 square millimeters at 200 watts, while the irreversible electroporation area reaches approximately 15 square millimeters at the same power level. The data demonstrates that both reversible and irreversible electroporation areas increase with increasing applied power, with the reversible electroporation area increasing at a faster rate than the irreversible electroporation area over the measured power range.

Referring to FIG. 6B, a dual-axis graph shows the difference between reversible and irreversible electroporation areas on the left vertical axis and the percent irreversible electroporation on the right vertical axis, both plotted against power in watts on the horizontal axis. A shaded region indicates an optimal range of power delivery, spanning approximately from 100 watts to 150 watts. Within this optimal range, the difference between reversible and irreversible areas is maximized while the percentage of irreversible electroporation remains relatively low.

With continued reference to FIG. 6B, the optimal power range corresponds to conditions favorable for gene electrotransfer applications where cell viability is desired. At power levels below the optimal range, the total electroporation area is limited, reducing the number of cells that can be transfected. At power levels above the optimal range, the percentage of irreversible electroporation increases, resulting in cell death rather than reversible electroporation suitable for gene delivery. By selecting a power level within the optimal range, reversible electroporation can be maximized while irreversible electroporation is minimized, thereby achieving efficient gene delivery while maintaining cell viability.

The data presented in FIG. 6A and FIG. 6B, obtained using the multi-needle electrode array configuration shown in FIG. 5, demonstrates that an optimal power range exists for gene electrotransfer applications. The six-needle electrode array configuration provides a substantially uniform power density distribution across the treatment region when operated within the optimal power range. The hydrogel tissue mimic with T-cells embedded at lymph node-relevant cellular density and collagen concentration allows for optimization of treatment protocols that can be translated to in vivo lymph node gene electrotransfer applications.

Referring to FIG. 7, a series of five circular cross-sectional views illustrates the progression of electroporation zones at increasing power levels from 40 W to 200 W. Each circular view represents a tissue cross-section with six electrode positions arranged around the periphery, corresponding to the six-needle electrode array configuration described with respect to FIG. 5. The progression demonstrates how increasing applied power correlates with expanding zones of electroporation, transitioning from minimal effect at lower power levels through reversible electroporation at intermediate power levels to irreversible electroporation at higher power levels.

At 40 W, the electrodes appear as small crescent-shaped regions with minimal effect on the surrounding tissue, which remains predominantly dark. The low power level produces insufficient power density within the tissue to exceed the threshold for reversible electroporation across substantial portions of the treatment region. At this power level, electroporation effects are limited to small regions immediately adjacent to the electrode surfaces where power density is highest.

With continued reference to FIG. 7, at 80 W, a central zone labeled rEP emerges, indicating a region of reversible electroporation that extends between the electrodes in a cross-shaped or star-shaped pattern. The increased power level produces power density values exceeding the reversible electroporation threshold across a larger portion of the tissue between the electrodes. The reversible electroporation zone at 80 W demonstrates that intermediate power levels can produce substantial regions of reversible electroporation suitable for gene electro transfer applications.

At 120 W, the reversible electroporation zone expands further, covering a larger central area while the regions immediately adjacent to the electrodes show increased effect. The power density distribution at 120 W produces reversible electroporation across a majority of the central treatment region. This power level falls within the optimal range identified in FIG. 6B, where the difference between reversible and irreversible electroporation areas is maximized.

As further shown in FIG. 7, at 160 W, the central zone continues to expand, with the reversible electroporation region occupying most of the central tissue area and darker zones remaining primarily at the electrode positions. The darker zones at the electrode positions indicate regions where power density exceeds the threshold for irreversible electroporation. At this power level, the irreversible electroporation zones begin to expand from the electrode surfaces into the surrounding tissue.

At 200 W, the central region is labeled IRE, indicating irreversible electroporation, where the affected zone has expanded to cover the majority of the tissue cross-section. The electrode regions appear as darker peripheral zones. At this highest power level, the power density throughout the central treatment region exceeds the threshold for irreversible electroporation, resulting in cell death rather than reversible electroporation. The progression from 40 W to 200 W demonstrates that power levels above the optimal range identified in FIG. 6B produce excessive irreversible electroporation that is unsuitable for gene electrotransfer applications where cell viability is desired.

The progression illustrated in FIG. 7 provides visual confirmation of the quantitative data presented in FIG. 6A and FIG. 6B. The transition from minimal electroporation at 40 W to reversible electroporation at 80 W and 120 W to irreversible electroporation at 160 W and 200 W corresponds to the curves showing increasing reversible and irreversible electroporation areas with increasing power. The optimal power range of approximately 100 W to 150 W identified in FIG. 6B corresponds to power levels between the 80 W and 160 W conditions shown in FIG. 7, where reversible electroporation zones are maximized while irreversible electroporation zones remain limited to regions immediately adjacent to the electrodes.

Referring to FIG. 8A, FIG. 8B, FIG. 8C, FIG. 8D, and FIG. 8E, a time-lapse series of fluorescence images illustrates fluid drainage pathways in lymph nodes following injection of a fluorescent solution. The series comprises five panels, each labeled with a timestamp indicating the elapsed time from the start of injection. FIG. 8A shows the initial state at zero seconds, with two outlined regions identified as PALN (proper axillary lymph node) and AALN (accessory axillary lymph node), both appearing dark prior to fluorescent solution distribution. A scale bar is present in FIG. 8A for reference.

With continued reference to FIG. 8B, the state at thirty seconds is shown, where fluorescence begins to appear within the lymph node structures. The fluorescent solution injected into the AALN begins to distribute within the lymph node tissue. FIG. 8C shows the state at sixty seconds, with increased fluorescence intensity visible in both lymph node regions. The progression from FIG. 8A through FIG. 8C demonstrates that injected substances distribute within the primary lymph node during the first minute following injection.

As further shown in FIG. 8D, the state at ninety seconds demonstrates further accumulation and spread of the fluorescent solution within the lymph node structures. FIG. 8E shows the state at one hundred twenty seconds, where the fluorescence distribution has reached a more extensive pattern throughout the lymph node structures. The progression of images from FIG. 8A through FIG. 8E demonstrates the flow and drainage of injected fluorescent solution from the AALN to the PALN over the two-minute observation period.

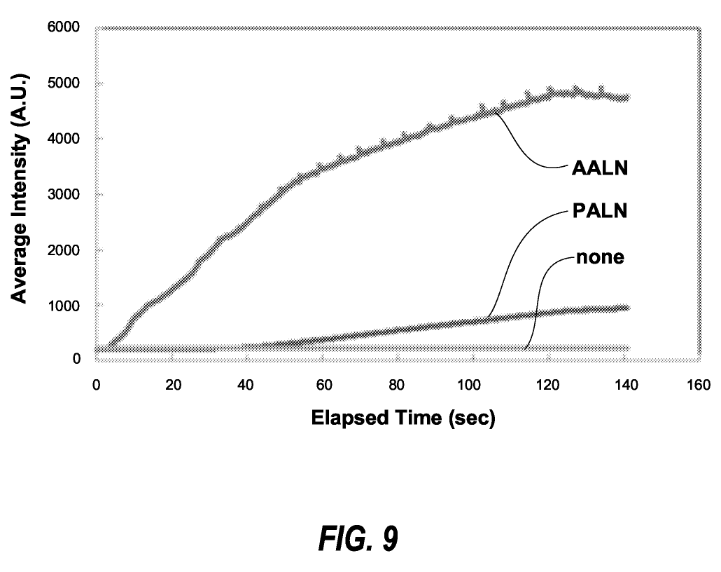

Referring to FIG. 9, a graph shows average intensity in arbitrary units as a function of elapsed time in seconds. Three curves are presented, each representing a different experimental condition. The uppermost curve, labeled AALN, rises steeply from approximately zero at time zero to approximately 3500 arbitrary units at around 60 seconds, then continues to increase more gradually, reaching a plateau of approximately 4700 arbitrary units by 120 seconds. The middle curve, labeled PALN, begins rising after a delay of approximately 40 seconds, increases gradually, and reaches approximately 1000 arbitrary units by 140 seconds. The lowest curve, labeled none, remains near zero throughout the entire measurement period from 0 to 160 seconds, representing regions outside the lymph nodes showing minimal to no fluorescence signal.

With continued reference to FIG. 9, the temporal relationship between fluorescence intensity measurements at different anatomical locations demonstrates that injected substances remain within the primary lymph node and subsequently drain to other lymph nodes. The AALN curve shows the fastest and highest intensity response, indicating that the injected fluorescent solution accumulates within the injection site lymph node. The PALN curve shows a delayed and lower intensity response, indicating that the fluorescent solution begins to flow from the primary lymph node to adjacent lymph nodes at approximately 40 to 60 seconds following injection. The data indicates that injected substances stay within the primary lymph node for at least 2 hours and start to flow to other lymph nodes at approximately 1 hour.

The fluid drainage pathway data presented in FIG. 8A through FIG. 8E and FIG. 9 informs the timing parameters for gene electrotransfer applications in lymph nodes. The method can include incubating the genetic material with the tissue for 30 minutes to 2 hours to allow for biodistribution throughout the tissue prior to delivering the pulsed electric field. In some cases, the method includes injecting the genetic material into the lymph node and maintaining for approximately 1 hour to allow mRNA to diffuse through the lymph node before applying electric pulses. The 1 hour incubation period provides sufficient time for maximal diffusion of mRNA within the primary lymph node without losing mRNA to diffusion to adjacent lymph nodes. The incubation period allows the genetic material to distribute throughout the lymph node structure so that T-cells throughout the lymph node can receive the genetic material during subsequent electroporation.

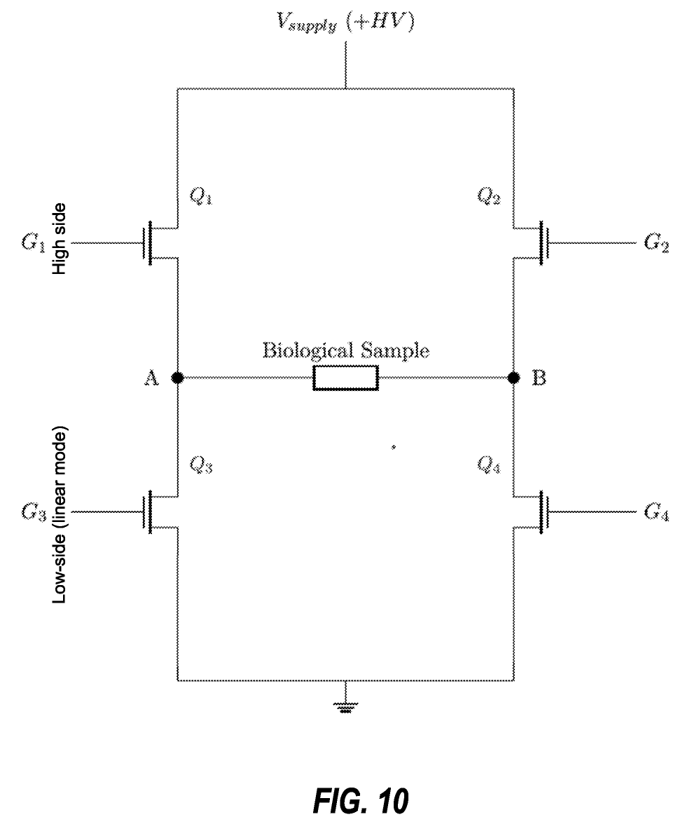

Referring to FIG. 10, a full-bridge topology circuit configured for delivering electrical pulses to a biological sample is illustrated. The circuit comprises a plurality of transistors arranged in an H-bridge configuration, with a high-voltage supply connected at the top and a ground connection at the bottom. The pulse generator can comprise a full-bridge topology including the plurality of transistors arranged in the H-bridge configuration.

The upper portion of the bridge includes two high-side transistors positioned on the left and right sides, each controlled by respective gate signals. The high-side transistors function as polarity switches, determining the direction of current flow through the load. The source terminals of both high-side transistors are connected to the positive high-voltage supply rail. The high-side transistors require galvanic isolation for their gate drivers because their source terminals swing up to the supply voltage during operation.

With continued reference to FIG. 10, the lower portion of the bridge includes two low-side transistors, also positioned on the left and right sides, each controlled by respective gate signals. At least one transistor of the plurality of transistors is configured to operate in a linear mode to regulate current flow through the biological tissue. The low-side transistors are configured to operate in linear mode, enabling continuous current regulation during pulse delivery. The source terminals of both low-side transistors are connected to a common ground reference. The low-side transistors are ground-referenced and can be driven directly by control circuitry such as an error amplifier.

A biological sample is positioned at the center of the bridge, connected between nodes on the left and right sides of the circuit. The left node connects the drain of the left high-side transistor to the drain of the left low-side transistor. Similarly, the right node connects the drain of the right high-side transistor to the drain of the right low-side transistor.

As further shown in FIG. 10, the circuit operates by activating diagonal pairs of transistors to drive current through the biological sample in either direction. When the left high-side transistor and the right low-side transistor are activated, current flows through the sample in one direction. Conversely, when the right high-side transistor and the left low-side transistor are activated, current flows in the opposite direction. This configuration enables the delivery of biphasic pulses to the biological sample.

The linear-mode operation of the low-side transistors allows for precise current regulation, maintaining constant current or constant power delivery to the biological sample despite variations in tissue impedance during electroporation. By operating in the linear region rather than switching between saturation and cutoff states, the low-side transistors can continuously adjust current flow in response to feedback signals from a sensing circuit.

In some cases, the pulse generator can comprise a Marx generator topology as an alternative to the H-bridge configuration for generating high voltage pulses. The Marx generator topology can charge multiple capacitors in parallel and discharge the capacitors in series to produce high voltage output pulses. The Marx generator topology can be combined with current regulation circuitry to achieve constant power delivery to biological tissue.

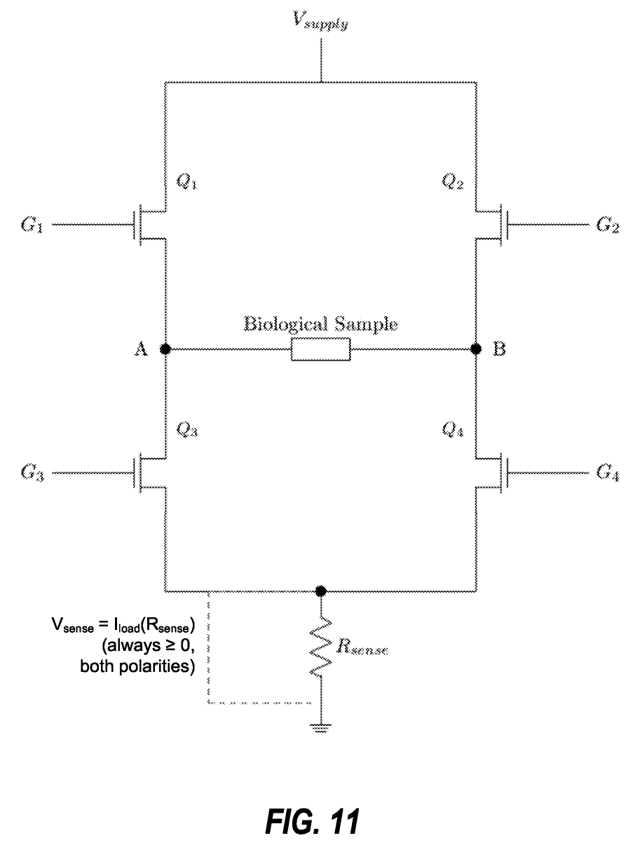

Referring to FIG. 11, a full-bridge topology circuit configured for delivering bidirectional current through a biological sample is illustrated. The circuit comprises four transistors arranged in an H-bridge configuration, with two high-side transistors positioned in the upper portion of the bridge and two low-side transistors positioned in the lower portion. Each transistor has an associated gate terminal for receiving control signals. A supply voltage is connected to the top of the bridge, providing power to the circuit.

The biological sample is positioned at the center of the bridge between two nodes, with current flowing through the sample in either direction depending on which diagonal pair of transistors is activated. When one diagonal pair is activated, current flows in one direction through the biological sample, and when the opposite diagonal pair is activated, current flows in the reverse direction. The high-side transistors function as polarity switches, determining the direction of current flow through the biological sample. The low-side transistors operate in linear mode to provide continuous current regulation.

With continued reference to FIG. 11, the sensing circuit comprises a sense resistor positioned in a common low-side return path of the full-bridge topology. The sense resistor is connected between the low-side transistors and ground. The sense resistor enables current sensing by developing a voltage proportional to the load current. Load current flows through the sense resistor in a same direction for both positive and negative pulse polarities. Because the load current flows through the sense resistor in the same direction regardless of which diagonal pair of transistors is active, the sense voltage remains positive for both pulse polarities.

As further shown in FIG. 11, this arrangement allows a single differential amplifier across the sense resistor to measure current for both polarities of operation. The differential amplifier can be configured with high bandwidth and high common-mode rejection ratio to accurately measure the voltage across the sense resistor. The sense voltage can be expressed as the product of the load current and the sense resistor value, and the sense voltage remains greater than or equal to zero for both pulse polarities. The circuit topology enables the delivery of controlled bidirectional pulses to the biological sample while providing continuous feedback of the delivered current through the sense resistor. The gate terminals of each transistor receive control signals to selectively activate the appropriate transistor pairs and regulate the current magnitude through linear operation of the low-side transistors.

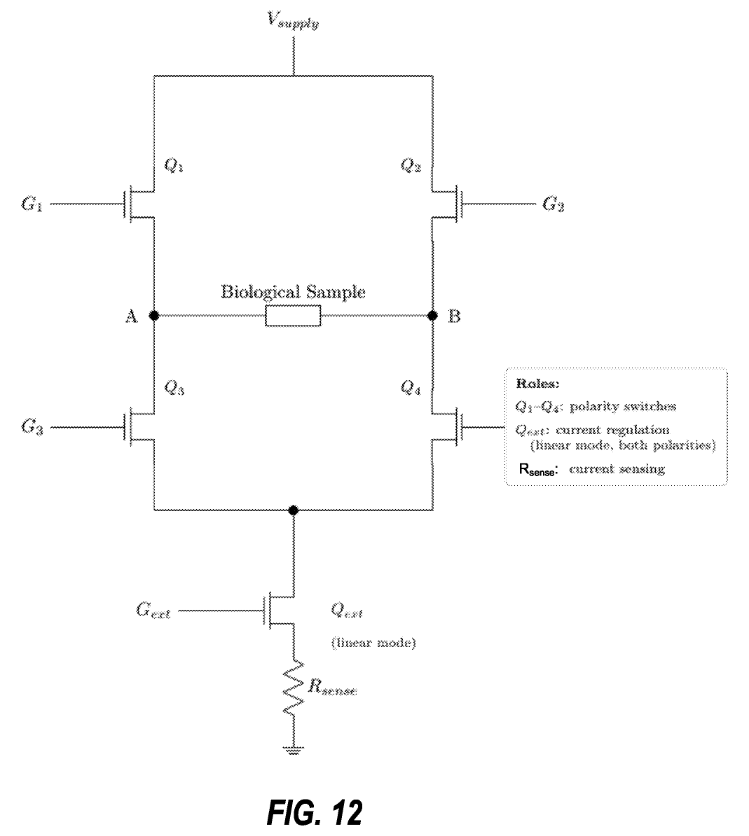

Referring to FIG. 12, a full-bridge topology circuit configured for delivering bidirectional current through a biological sample is illustrated. The circuit comprises a supply voltage connected to two high-side transistors, which serve as polarity switches. The high-side transistors are controlled by respective gate signals and are positioned at the upper portion of the bridge configuration. Two low-side transistors are positioned at the lower portion of the bridge, also controlled by respective gate signals. The biological sample is connected between nodes positioned at the midpoints of the two vertical branches of the bridge, such that current flows through the sample when diagonal pairs of transistors are activated.

With continued reference to FIG. 12, the circuit includes an external transistor positioned in a common low-side return path below the junction of the two low-side transistors. The external transistor operates in linear mode and provides current regulation for both pulse polarities through a single fixed, ground-referenced feedback path. A gate signal controls the external transistor to modulate current flow through the biological sample. Operating a transistor in a linear mode to regulate current flow through the biological tissue enables precise control of the current magnitude delivered to the biological sample.

As further shown in FIG. 12, the bridge transistors act as polarity-selection switches driven fully between saturation and cutoff states. The high-side transistors and the low-side transistors of the H-bridge configuration function to select the direction of current flow through the biological sample without providing current regulation. When one diagonal pair of bridge transistors is activated, current flows in one direction through the biological sample, and when the opposite diagonal pair is activated, current flows in the reverse direction. The external transistor in the common return path provides continuous current regulation in linear mode, allowing precise control of current magnitude independent of the selected polarity.

The circuit configuration shown in FIG. 12 differs from the configuration shown in FIG. 11 in that the current regulation function is separated from the polarity selection function. In FIG. 11, the low-side transistors of the H-bridge provide both polarity selection and current regulation by operating in the linear region. In FIG. 12, the bridge transistors provide polarity selection by switching between saturation and cutoff, while the external transistor provides current regulation by operating in the linear region. This separation can simplify the feedback architecture because the external transistor remains in linear mode for both pulse polarities, eliminating the need to switch the current regulation function between different transistors when the pulse polarity changes.

With continued reference to FIG. 12, a sense resistor is positioned between the external transistor and ground, enabling current sensing in the common low-side return path. Load current flows through the sense resistor in the same direction for both pulse polarities, producing a voltage across the sense resistor that is proportional to the load current and remains non-negative regardless of the polarity of current through the biological sample. A single differential amplifier across the sense resistor can monitor current for both polarities, simplifying the feedback architecture while maintaining bidirectional current capability.

The high-side transistors require galvanic isolation for their gate drivers because their source terminals swing up to the supply voltage during operation. Galvanically isolated gate drivers provide the voltage level shifting and isolation barrier between the ground-referenced control circuitry and the floating high-side transistor gate terminals. The low-side transistors and the external transistor are ground-referenced and can be driven directly by an error amplifier without requiring galvanic isolation. The error amplifier continuously adjusts the gate of the external transistor to null the error between a reference current and the measured load current, maintaining constant current despite changes in load voltage as the membrane capacitor charges during electroporation.

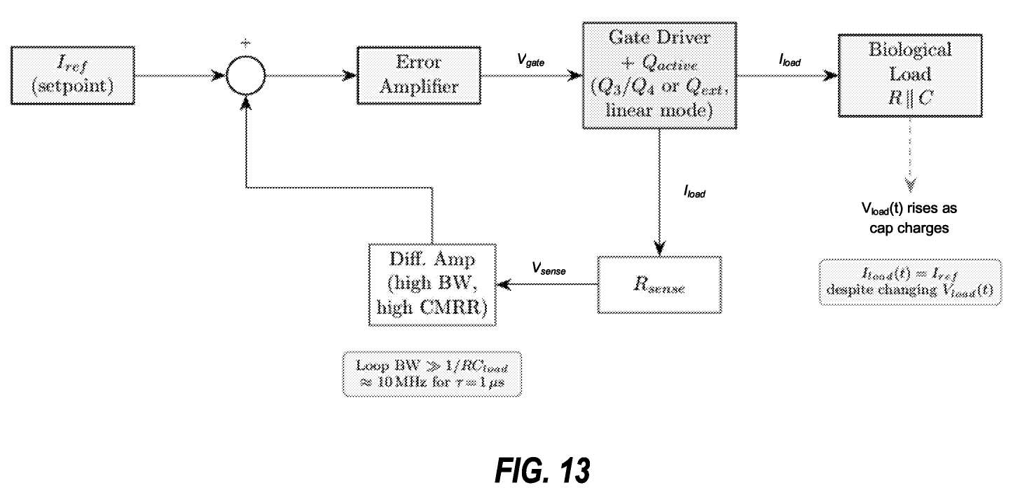

Referring to FIG. 13, a block diagram illustrates a current feedback loop for constant-current electroporation control. The system includes a reference current setpoint input, a summing junction, an error amplifier, a gate driver with an active transistor operating in linear mode, a biological load, a sense resistor, and a differential amplifier. The reference current setpoint provides the desired current value to the summing junction, which compares the reference current setpoint with a feedback signal representing the measured load current.

With continued reference to FIG. 13, the error amplifier receives the difference between the reference current and the measured current from the summing junction and generates a gate voltage signal. The error amplifier continuously adjusts the gate of the active linear transistor to null the error between reference current and load current. The gate driver and active transistor block receives the gate voltage and controls current delivery to the biological load. The active transistor, which can be one of the low-side transistors of an H-bridge or an external transistor depending on the embodiment as described with respect to FIG. 11 and FIG. 12, operates in linear mode to provide continuous current regulation.

As further shown in FIG. 13, the biological load is modeled as a parallel combination of resistance and capacitance, representing the electrical characteristics of tissue during electroporation. The parallel resistance represents the ionic conductivity of the tissue, while the parallel capacitance represents the membrane capacitance of cells within the tissue. As current flows through the biological load, the load voltage rises as the capacitive component charges. The time constant of the biological load can be approximately 1 microsecond, determined by the product of the load resistance and the load capacitance.

The load current flows through the sense resistor, which generates a sense voltage proportional to the current magnitude. The differential amplifier, configured with high bandwidth and high common-mode rejection ratio, measures the voltage across the sense resistor and provides the feedback signal to the summing junction. The feedback loop bandwidth is specified to be much greater than the inverse of the load time constant. For a time constant of approximately 1 microsecond, the feedback loop bandwidth can be approximately 10 megahertz or greater. The feedback loop implements a bandwidth exceeding 1/RC_{load} to maintain current regulation despite the rising load voltage as the membrane capacitor charges. This high bandwidth ensures that the system can maintain constant current despite changes in load voltage during electroporation.

With continued reference to FIG. 13, the feedback loop maintains the load current equal to the reference current regardless of variations in load voltage over time. As the membrane capacitor charges during pulse delivery, the load voltage increases. Without feedback control, the increasing load voltage would cause the current to decrease if a constant voltage were applied. The feedback loop compensates for the rising load voltage by increasing the voltage applied by the pulse generator to maintain the current at the reference setpoint. The error amplifier detects any deviation between the reference current and the measured load current and adjusts the gate voltage of the active transistor to correct the deviation.

The controller can implement dead time control where both low-side transistors are driven into saturation to short the output to zero. During dead time, the membrane capacitor discharges through the load resistance before the next pulse of opposite polarity. The time constant for membrane capacitor discharge can be approximately 1 microsecond. The dead time interval allows the membrane capacitor to discharge to a baseline voltage level, preventing charge accumulation that could affect subsequent pulses. Following the dead time interval, the next pulse of opposite polarity can be delivered by activating the opposite diagonal pair of transistors in the H-bridge configuration.

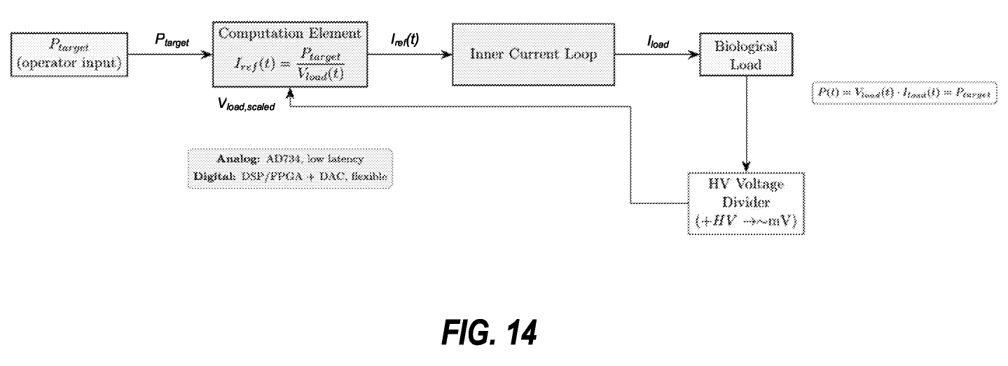

Referring to FIG. 14, a block diagram illustrates a constant power control system for electroporation. The system includes an operator input for a target power value, a computation element, an inner current loop, a biological load, and a high voltage divider. The operator input provides a target power value to the computation element. The computation element receives a scaled load voltage from the high voltage divider as feedback to perform calculations for determining a reference current setpoint.

With continued reference to FIG. 14, the controller implements constant power control using an outer loop where the computation element sets the reference current as the target power divided by the instantaneous load voltage. The computation element calculates a reference current as a function of time by dividing the target power by the load voltage as a function of time. The reference current output from the computation element is provided to the inner current loop, which regulates the current delivered to the biological load as described previously with respect to FIG. 13. The biological load receives the load current from the inner current loop, and the instantaneous power delivered to the biological load equals the product of the load voltage and load current, which equals the target power.

As further shown in FIG. 14, the high voltage divider scales down the high voltage present at the biological load to a millivolt range suitable for feedback to the computation element. The high voltage divider provides a scaled representation of the instantaneous load voltage that the computation element uses to calculate the reference current setpoint in real-time.

The controller can be configured to calculate an instantaneous power as a product of the measured voltage and the measured current. The controller can adjust the current delivered by the pulse generator to maintain the instantaneous power at a target power value by dividing the target power value by the measured voltage to determine a reference current setpoint. As described previously, adjusting at least one of the voltage or the current can comprise calculating an instantaneous power as a product of the monitored voltage and the monitored current and modifying the current to maintain the instantaneous power at a target power value.

With continued reference to FIG. 14, the controller can comprise an analog divider configured to calculate a reference current setpoint in real-time. The computation element for constant power control can be implemented using an analog divider for low latency operation. In some cases, the analog divider comprises an AD734 analog divider integrated circuit. The analog divider implementation provides low latency calculation of the reference current setpoint, enabling rapid response to changes in load voltage during pulse delivery.

Alternatively, the controller can comprise a digital processor configured to calculate the reference current setpoint using a digital-to-analog converter. The computation element for constant power control can be implemented using a digital processor such as a DSP (digital signal processor) or FPGA (field-programmable gate array). The digital processor implementation provides flexible calculation of the reference current setpoint and can accommodate complex control algorithms. The digital processor receives digitized voltage measurements, calculates the reference current setpoint by dividing the target power by the measured voltage, and outputs the reference current setpoint through a digital-to-analog converter to the inner current loop.

The outer loop architecture enables constant power delivery to the biological load despite changes in load impedance during electroporation. As the load impedance changes during electroporation due to membrane permeabilization and tissue heating, the load voltage changes for a given current. The computation element automatically adjusts the current setpoint to maintain the product of voltage and current at the target power level. By continuously dividing the target power by the instantaneous load voltage, the computation element generates a reference current setpoint that, when regulated by the inner current loop, produces constant power delivery to the biological tissue regardless of impedance variations.

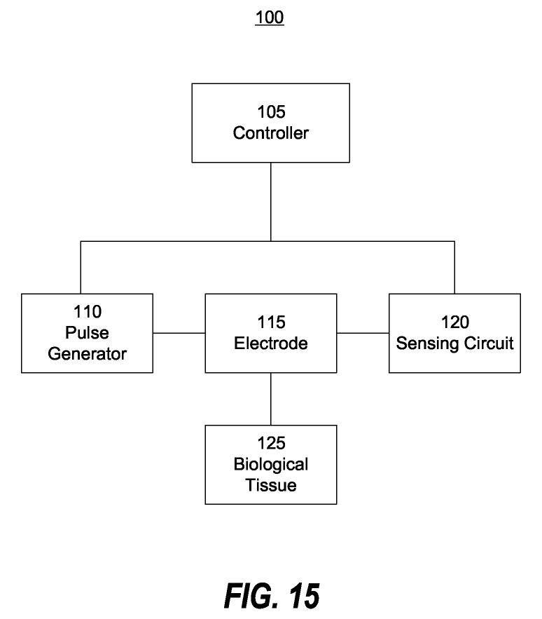

Referring to FIG. 15, a system 100 for delivering pulsed electric fields to biological tissue 125 is illustrated. The system 100 includes a controller 105, a pulse generator 110, an electrode 115, and a sensing circuit 120. The controller 105 is positioned at the top of the diagram and is connected to the pulse generator 110, the electrode 115, and the sensing circuit 120, which are arranged in a row below the controller 105. The electrode 115 is centrally positioned and is connected to the biological tissue 125, which is shown at the bottom of the diagram.

With continued reference to FIG. 15, the electrode 115 is configured to be positioned in proximity to the biological tissue 125 to facilitate delivery of a pulsed electric field. As described previously with respect to FIG. 5, the electrode 115 can comprise a plurality of needle electrodes arranged in an array configuration. The electrode 115 can also comprise catheter-based electrodes for cardiac applications or surface electrodes for various tissue treatment applications.

The pulse generator 110 is electrically coupled to the electrode 115 and is configured to deliver a pulsed electric field to the biological tissue 125 through the electrode 115. As described previously with respect to FIG. 10, FIG. 11, and FIG. 12, the pulse generator 110 can comprise a full-bridge topology including a plurality of transistors arranged in an H-bridge configuration. The pulse generator 110 delivers the pulsed electric field to the biological tissue 125 to induce electroporation, which can be reversible electroporation for gene delivery applications or irreversible electroporation for tissue ablation applications.

As further shown in FIG. 15, the sensing circuit 120 is configured to measure a voltage and a current delivered to the biological tissue 125 during delivery of the pulsed electric field. As described previously with respect to FIG. 11 and FIG. 12, the sensing circuit 120 can comprise a sense resistor positioned in a common low-side return path of the full-bridge topology. The sensing circuit 120 provides continuous feedback of the delivered current and voltage to enable real-time power monitoring and control.

The controller 105 is coupled to the sensing circuit 120 and the pulse generator 110. The controller 105 receives measurements from the sensing circuit 120 and adjusts at least one of the voltage or the current delivered by the pulse generator 110 to maintain a substantially constant power delivered to the biological tissue 125. As described previously with respect to FIG. 14, the controller 105 can calculate an instantaneous power as a product of the measured voltage and the measured current and can adjust the current delivered by the pulse generator 110 to maintain the instantaneous power at a target power value. The substantially constant power produces a substantially constant power density distribution within the biological tissue 125 that is independent of electrical conductivity variations in the biological tissue 125.

With continued reference to FIG. 15, the controller 105 actively monitors current and lowers voltage to maintain a specified power range and account for tissue property changes due to heating and electroporation. During delivery of the pulsed electric field, tissue electrical properties can change as a result of membrane permeabilization and Joule heating. The controller 105 compensates for these tissue property changes by continuously adjusting the voltage or current to maintain the target power level.

The system 100 can monitor tissue bioimpedance to gauge electroporation outcomes and determine when cells have been electroporated. The sensing circuit 120 can measure changes in tissue impedance during pulse delivery, where decreasing impedance indicates increasing membrane permeabilization. The controller 105 can analyze bioimpedance measurements to determine an electroporation status of the biological tissue 125 and can terminate delivery of the pulsed electric field based on the electroporation status indicating a predetermined level of electroporation has been achieved.

As further shown in FIG. 15, the system 100 can monitor tissue bioimpedance to detect electroporation saturation, heating, and thermal damage indicators. Electroporation saturation occurs when additional pulses produce minimal further increase in membrane permeabilization. Heating indicators can include changes in tissue conductivity associated with temperature rise. Thermal damage indicators can include impedance changes characteristic of protein denaturation or tissue coagulation. The controller 105 can use these bioimpedance-derived indicators to adjust treatment parameters or terminate pulse delivery to prevent undesired tissue damage.

The system 100 uses power monitoring to control Joule heating, which is linearly proportional to power, for minimizing side effects or complications when performing high pulse treatments near sensitive structures. By maintaining constant power delivery, the system 100 can achieve predictable and controlled Joule heating within the biological tissue 125. The controller 105 can limit the target power value to maintain tissue temperature below a threshold associated with thermal damage, thereby enabling electroporation treatments in proximity to thermally sensitive anatomical structures such as nerves, blood vessels, or cardiac conduction pathways.

The method can include delivering a preliminary low-voltage pulse to the biological tissue prior to delivering the pulsed electric field. The preliminary low-voltage pulse does not induce significant electroporation effects within the biological tissue. The preliminary low-voltage pulse can have a voltage magnitude and duration selected to avoid exceeding the reversible electroporation threshold of the biological tissue. By applying a voltage that does not induce electroporation, the electrical response of the biological tissue reflects the baseline electrical properties of the tissue without modification from membrane permeabilization.

The method can include measuring a response to the preliminary low-voltage pulse to determine a bulk electrical conductivity of the biological tissue. The response to the preliminary low-voltage pulse can include a current measurement corresponding to the applied voltage. The bulk electrical conductivity of the biological tissue can be calculated from the measured current and the applied voltage using Ohm's law, where conductivity is proportional to the ratio of current to voltage for a given electrode geometry. The electrode geometry factor can be determined through calibration or computational modeling for the specific electrode configuration being used.

The method can include calculating an initial voltage or current setting based on the determined bulk electrical conductivity to achieve a target power. Once the bulk electrical conductivity of the biological tissue is determined from the preliminary low-voltage pulse response, the voltage or current setting for the therapeutic pulsed electric field can be calculated to deliver the target power to the biological tissue. The target power can be selected to produce a power density within the range from 1,000 W/cm³ to 10,000 W/cm³ as described previously. The initial voltage setting can be calculated by determining the voltage that, when applied to tissue having the measured bulk electrical conductivity, produces the target power. Alternatively, the initial current setting can be calculated by determining the current that, when combined with the expected voltage drop across tissue having the measured bulk electrical conductivity, produces the target power.

The preliminary low-voltage pulse approach allows for determination of tissue electrical properties prior to therapeutic pulse delivery without requiring a priori information about the target tissue. The calculated initial voltage or current setting provides a starting point for the constant-power control system, which can then make real-time adjustments during therapeutic pulse delivery to maintain the target power as tissue properties change due to heating and electroporation effects. There can be slight differences between the calculated power and the delivered power due to further decreases in tissue resistance following electroporation, but the preliminary measurement allows estimation of the power within an acceptable range for initiating treatment.

The pulsed electric field can comprise monophasic pulses, biphasic pulses, or a combination thereof. Monophasic pulses consist of pulses having a single polarity, where current flows in one direction through the biological tissue during each pulse. Biphasic pulses consist of pulses having alternating polarities, where current flows in one direction during a first phase and in the opposite direction during a second phase. The H-bridge configurations described previously with respect to FIG. 10, FIG. 11, and FIG. 12 enable delivery of both monophasic and biphasic pulses by selectively activating diagonal pairs of transistors. For monophasic pulse delivery, the same diagonal pair of transistors is activated for each pulse. For biphasic pulse delivery, alternating diagonal pairs of transistors are activated to produce pulses of opposite polarity. A combination of monophasic and biphasic pulses can be delivered within a single treatment protocol, where some pulses are monophasic and other pulses are biphasic.

The pulsed electric field can comprise pulses having a pulse duration in a range from 500 nanoseconds to 100 microseconds. Pulse durations within this range are associated with electroporation mechanisms where the applied electric field induces membrane permeabilization. Pulse durations shorter than 500 nanoseconds can involve different biophysical mechanisms including intracellular effects and oxidation processes. Pulse durations within the 500 nanosecond to 100 microsecond range are used in cardiac pulsed field ablation and oncology applications. The pulse duration can be selected based on the target tissue type and the desired electroporation outcome, whether reversible electroporation for gene delivery or irreversible electroporation for tissue ablation.

For gene electrotransfer applications, the pulsed electric field can comprise pulses having a pulse duration in a range from 100 microseconds to 40,000 microseconds. Longer pulse durations within this range can facilitate electrophoretic movement of charged molecules such as DNA and mRNA across the permeabilized cell membrane. The extended pulse duration provides additional time for genetic material to migrate through membrane pores formed during electroporation. Pulse durations of 100 microseconds to 40,000 microseconds have been used in previously optimized T-cell transfection protocols in low conductivity buffer.

The pulsed electric field can comprise 1 to 8 series of pulses for T-cell transfection protocols. Each series can include multiple individual pulses delivered in sequence. The number of pulse series can be selected based on the desired transfection efficiency and the tolerance of the target cells to repeated electroporation. Multiple pulse series can increase the total number of cells that are transfected while allowing recovery time between series to maintain cell viability.

The pulsed electric field can be delivered at an electric field strength of 750 to 1500 V/cm in tissue for T-cell transfection. Electric field strengths within this range have been used in previously optimized T-cell transfection protocols. The electric field strength can be selected in combination with the pulse duration and number of pulse series to achieve the desired balance between transfection efficiency and cell viability. For constant-power electroporation, the electric field strength within the tissue varies with tissue electrical conductivity, but the power density remains substantially constant as described previously. The electric field strength range of 750 to 1500 V/cm corresponds to power density values within the range from 1,000 W/cm³ to 10,000 W/cm³ for typical tissue conductivity values encountered in lymph node tissue.

The method can include monitoring a bioimpedance of the biological tissue during delivery of the pulsed electric field to determine an electroporation status of the biological tissue. Bioimpedance measurements provide real-time information about the electrical properties of the biological tissue as electroporation progresses. During electroporation, the formation of pores in cell membranes increases membrane permeability, which results in measurable changes in tissue impedance. The bioimpedance of the biological tissue can be monitored by measuring the voltage and current delivered to the tissue and calculating the impedance as the ratio of voltage to current. Changes in bioimpedance during pulse delivery indicate changes in membrane permeabilization state.

The electroporation status of the biological tissue can be determined from bioimpedance measurements by analyzing the magnitude and rate of impedance change during pulse delivery. As cells within the biological tissue become electroporated, the tissue impedance decreases due to increased ionic conductivity through permeabilized membranes. The rate of impedance decrease can indicate the rate at which cells are being electroporated. A plateau in impedance decrease can indicate that electroporation saturation has been reached, where additional pulses produce minimal further increase in membrane permeabilization. The electroporation status can also be determined by comparing measured bioimpedance values to reference values associated with known electroporation states for the target tissue type.

The method can include terminating delivery of the pulsed electric field based on the electroporation status indicating a predetermined level of electroporation has been achieved. The predetermined level of electroporation can be defined as a target impedance value, a target percentage decrease in impedance from baseline, or a target rate of impedance change. When the bioimpedance measurements indicate that the predetermined level of electroporation has been achieved, the controller can terminate pulse delivery to prevent excessive electroporation that could result in irreversible cell damage. For gene electrotransfer applications where cell viability is desired, terminating pulse delivery when the predetermined level of reversible electroporation has been achieved can maximize transfection efficiency while minimizing cell death.

The predetermined level of electroporation can be tissue-specific and can be determined through calibration experiments for the target tissue type. For lymph node gene electrotransfer applications, the predetermined level of electroporation can correspond to a bioimpedance change associated with sufficient membrane permeabilization for mRNA uptake by T-cells while maintaining T-cell viability. The predetermined level can also account for the specific genetic material being delivered, as larger molecules can require greater membrane permeabilization for efficient uptake compared to smaller molecules.

Bioimpedance monitoring can also be used to detect indicators of heating and thermal damage during pulse delivery. As tissue temperature increases due to Joule heating, tissue conductivity increases, which manifests as a decrease in tissue impedance. The rate and magnitude of impedance decrease associated with heating can be distinguished from impedance changes associated with electroporation based on the temporal characteristics of the impedance change. Rapid impedance changes occurring during individual pulses can indicate electroporation effects, while gradual impedance changes occurring over multiple pulses can indicate heating effects. When bioimpedance measurements indicate that thermal damage thresholds are being approached, the controller can terminate pulse delivery or reduce the target power to prevent thermal injury to the biological tissue.

The method for in situ transfection of T-cells within a lymph node includes controlling a power delivered to the lymph node during delivery of the pulsed electric field to maintain a substantially constant power density within the lymph node. As described previously with respect to FIG. 3C and FIG. 4B, maintaining a substantially constant power density produces consistent reversible electroporation outcomes regardless of variations in tissue electrical conductivity. The substantially constant power density within the lymph node facilitates uptake of the genetic material by the T-cells residing within the lymph node tissue.

Controlling the power delivered to the lymph node comprises monitoring a voltage and a current delivered to the lymph node during delivery of the pulsed electric field. The voltage and current can be monitored continuously throughout each pulse using sensing circuitry as described previously. The monitored voltage represents the instantaneous voltage drop across the lymph node tissue between the electrodes. The monitored current represents the instantaneous current flowing through the lymph node tissue during pulse delivery.

Controlling the power delivered to the lymph node further comprises calculating an instantaneous power as a product of the monitored voltage and the monitored current. The instantaneous power calculation can be performed in real-time during pulse delivery using analog circuitry or digital processing as described previously. The instantaneous power represents the rate of energy delivery to the lymph node tissue at each moment during the pulsed electric field delivery.

Controlling the power delivered to the lymph node further comprises adjusting at least one of the voltage or the current to maintain the instantaneous power at a target power value corresponding to a power density in a range from 1,000 W/cm³ to 10,000 W/cm³. The target power value can be selected based on the electrode geometry and the desired power density within the lymph node tissue. When the calculated instantaneous power deviates from the target power value, the voltage or current can be adjusted to return the instantaneous power to the target value. The adjustment can be performed by modifying the current while allowing the voltage to vary with tissue impedance, or by modifying the voltage while allowing the current to vary, or by modifying both voltage and current in coordination.

The genetic material delivered to the lymph node can comprise modified CleanCap mRNA with N1-Methylpseudouridine modification. The N1-Methylpseudouridine modification is designed to prevent immune recognition of the mRNA by immune cells within the lymph node. The modification also decreases binding of proteases to the mRNA, providing stability in tissues. The modified mRNA can remain stable within the lymph node tissue for at least 24 hours following injection, allowing sufficient time for biodistribution throughout the lymph node structure prior to electroporation and for subsequent translation of the mRNA into protein following cellular uptake.

The genetic material can be delivered at a concentration of 0.1 to 0.5 mg/ml throughout the tissue. The concentration can be selected based on the desired transfection efficiency and the volume of the lymph node being treated. Higher concentrations within the range can increase the amount of genetic material available for uptake by each electroporated T-cell. The genetic material can be diluted in RNase-free saline or low conductivity buffer prior to injection into the lymph node. The injection volume can be selected to achieve the target concentration throughout the lymph node tissue volume while avoiding excessive tissue distension that could affect electroporation outcomes.

The genetic material for in situ transfection of T-cells within a lymph node can comprise mRNA encoding a chimeric antigen receptor. As described previously, uptake of the mRNA by the T-cells produces autologous CAR T-cells within the lymph node. The mRNA encoding the chimeric antigen receptor can encode various CAR constructs targeting different tumor antigens, including CD19 for B-cell malignancies, BCMA for multiple myeloma, and other tumor-associated antigens. The CAR construct can be of various generations, including first generation CARs comprising an antigen-binding domain and a signaling domain, second generation CARs comprising an additional costimulatory domain, third generation CARs comprising multiple costimulatory domains, fourth generation CARs comprising cytokine-inducing capabilities, and fifth generation CARs comprising additional signaling modifications.

The mRNA can further encode a suicide gene under a T-cell specific promoter. The suicide gene provides a safety mechanism that allows clinicians to eliminate CAR T-cells if excessive proliferation, activation, or adverse effects occur following treatment. When the suicide gene is activated by administration of a corresponding prodrug or inducing agent, the CAR T-cells expressing the suicide gene undergo non-immunogenic apoptosis. The suicide gene can comprise herpes simplex virus thymidine kinase, inducible caspase 9, or other genes that induce cell death upon activation.

The T-cell specific promoter comprises a dLck promoter or a CD3δ promoter. The dLck promoter and the CD3δ promoter are well-characterized T-cell specific promoters that drive gene expression selectively within T-cells. By placing the suicide gene under a T-cell specific promoter, expression of the suicide gene is limited to T-cells that have taken up the genetic material, preventing expression in other cell types within the lymph node that can also be electroporated during treatment. The T-cell specific promoter provides an additional layer of safety by ensuring that the suicide gene function is restricted to the intended target cell population.

The genetic material can comprise mRNA encoding engineered T-cell receptors for generating T-cells with engineered TCRs. Engineered T-cell receptors can recognize intracellular tumor antigens presented on major histocompatibility complex molecules, providing an alternative approach to CAR T-cells for targeting tumor cells. The mRNA encoding engineered T-cell receptors can be delivered to T-cells within the lymph node using the constant-power electroporation techniques described herein.

The genetic material can comprise plasmid DNA for gene electrotransfer. Plasmid DNA can encode therapeutic genes, reporter genes, or regulatory elements for expression within target cells. Plasmid DNA delivered via electroporation does not integrate into the genome in most cases, resulting in transient expression of the encoded gene. Approximately 1-5% of cells transfected with plasmid DNA can permanently express the gene through random integration events. For applications where transient expression is desired, such as CAR T-cell generation where temporary CAR expression within the body is preferred, the non-integrating nature of plasmid DNA delivery via electroporation provides an advantage.

The genetic material can comprise siRNA for gene electrotransfer. Small interfering RNA can be delivered to target cells via electroporation to achieve gene knockdown through RNA interference mechanisms. The siRNA can target genes involved in immune checkpoint pathways, T-cell exhaustion, or other regulatory pathways to enhance T-cell function following transfection.

The genetic material can comprise proteins for delivery via electroporation. Proteins including transcription factors, enzymes, and antibodies can be delivered directly into cells through electroporation-induced membrane permeabilization. Protein delivery via electroporation provides immediate functional activity within target cells without requiring transcription and translation steps.

The genetic material can comprise CRISPR/Cas9 ribonucleoproteins for genome editing. CRISPR/Cas9 ribonucleoproteins consist of Cas9 protein complexed with guide RNA and can be delivered to target cells via electroporation to achieve site-specific genome editing. The CRISPR/Cas9 system can insert DNA sequences, delete sequences, or modify existing sequences within the target cell genome. For permanent CAR expression, CRISPR/Cas9 ribonucleoproteins can be used to insert CAR-encoding sequences into the T-cell genome at specific loci.

The genetic material can comprise CRISPR technology carrying insertion for a gene with T-cell specific promoters dLck or CD3δ for selective expression within T-cells. By inserting the CAR gene or other therapeutic gene under control of a T-cell specific promoter, expression of the inserted gene is restricted to T-cells even if other cell types within the lymph node receive the CRISPR components during electroporation. The selective expression within T-cells increases safety for clinical use by preventing expression of the therapeutic gene in non-target cell populations.

The genetic material can comprise a drug for delivery via electroporation. Chemotherapeutic agents, small molecule inhibitors, and other pharmaceutical compounds can be delivered into cells through electroporation-induced membrane permeabilization. Drug delivery via electroporation can achieve higher intracellular drug concentrations compared to passive diffusion or receptor-mediated uptake.

The genetic material can comprise a fluorescent dye for delivery via electroporation. Fluorescent dyes including calcein, propidium iodide, and Yo-Pro-1 can be delivered into cells via electroporation for visualization and quantification of electroporation outcomes. Fluorescent dye delivery can be used to assess reversible and irreversible electroporation areas in experimental systems.

The genetic material can comprise nanoparticles or lipid nanoparticles for delivery via electroporation. Nanoparticles and lipid nanoparticles can encapsulate genetic material or other therapeutic agents for delivery into cells. Electroporation can enhance cellular uptake of nanoparticles by increasing membrane permeability. The combination of nanoparticle encapsulation and electroporation-mediated delivery can provide advantages including protection of genetic material from degradation and enhanced intracellular delivery efficiency.

The electrode can comprise thin needles specifically designed for placement within lymph nodes. Thin needle electrodes can be inserted directly into lymph node tissue to deliver pulsed electric fields to T-cells residing within the lymph node structure. The thin needle configuration allows for minimally invasive access to lymph nodes while providing sufficient electrode surface area for current delivery. The needle electrodes can be arranged in arrays as described previously, with multiple needles positioned around the periphery of the lymph node to produce a substantially uniform power density distribution across the treatment region. The thin needle design accommodates the anatomical constraints of lymph node tissue, which can range from 5 mm to 15 mm in diameter in humans. The needle electrodes can be fabricated from biocompatible conductive materials suitable for contact with lymphatic tissue.

The electrode can comprise conductive hydrogel electrodes for inducing microscale electroporation. Conductive hydrogel electrodes provide conformal contact with tissue surfaces and can distribute current over larger areas compared to rigid metal electrodes. The conductive hydrogel material can conform to irregular tissue geometries, providing consistent electrode-tissue contact across the treatment region. Conductive hydrogel electrodes have been used for CRISPR delivery to lymph nodes, demonstrating proof-of-concept for engineering T-cells within lymph nodes to combat systemic diseases. The conductive hydrogel electrodes can induce microscale electroporation patterns that differ from the electroporation patterns produced by needle electrodes or plate electrodes. The microscale electroporation patterns can provide advantages for certain applications including reduced thermal effects and more uniform cellular uptake of genetic material.

The electrode can comprise a single probe with a surface electrode configuration. In the single probe with surface electrode configuration, one electrode is positioned within or adjacent to the target tissue while a second electrode is positioned on the tissue surface at a distance from the first electrode. Current flows from the single probe through the target tissue to the surface electrode, producing an electric field distribution that extends from the probe location toward the surface electrode. The single probe with surface electrode configuration can be used for treating tissues that are accessible from one direction, where insertion of multiple needle electrodes is not feasible or desirable. The surface electrode can comprise a conductive pad, a conductive gel electrode, or other electrode configuration suitable for contact with tissue surfaces.

The electrode can comprise a catheter configuration for treatment in the heart. Catheter-based electrodes can be advanced through blood vessels to reach cardiac tissue for pulsed field ablation applications. The catheter configuration allows for minimally invasive access to cardiac structures including the pulmonary veins, left atrium, and other cardiac chambers. Cardiac catheters can incorporate multiple electrodes arranged along the catheter body or at the catheter tip to deliver pulsed electric fields to cardiac tissue. The constant-power electroporation techniques described herein can be applied using catheter-based electrodes to achieve consistent ablation outcomes despite variations in cardiac tissue electrical properties. Cardiac tissue electrical properties can vary between patients and between different regions of the heart within the same patient, making constant-power delivery advantageous for achieving predictable treatment outcomes. The catheter configuration can accommodate the anatomical constraints of the cardiovascular system while providing sufficient electrode surface area for therapeutic energy delivery.

The electrode can comprise a single needle with an external grounding pad for monopolar or pseudo-monopolar treatment configurations. In the monopolar configuration, a single needle electrode is positioned within or adjacent to the target tissue, and an external grounding pad is positioned on the patient's skin at a location distant from the treatment site. Current flows from the single needle electrode through the patient's body to the external grounding pad. The monopolar configuration produces an electric field distribution that extends radially from the needle electrode toward the grounding pad. The pseudo-monopolar configuration is similar to the monopolar configuration but can involve multiple needle electrodes that are activated together as a single pole relative to the external grounding pad. The monopolar and pseudo-monopolar configurations can be used for treating deep tissues where positioning multiple electrodes around the target tissue is not feasible. The constant-power electroporation techniques described herein can compensate for patient-to-patient variability in the monopolar and pseudo-monopolar configurations, where variability in patient body composition, tissue thickness, and tissue electrical properties between the needle electrode and the grounding pad can affect treatment outcomes under constant voltage conditions. The external grounding pad can comprise a large-area conductive electrode designed to distribute return current over a sufficient area to prevent skin burns or discomfort at the grounding pad location.

The biological tissue can comprise skin tissue for in situ vaccine production. The constant-power electroporation techniques described herein can be applied to skin tissue to deliver genetic material encoding antigens for vaccine applications. Skin tissue contains antigen-presenting cells including Langerhans cells and dermal dendritic cells that can process and present antigens to initiate immune responses. By delivering mRNA or plasmid DNA encoding pathogen antigens or tumor antigens to skin tissue via constant-power electroporation, in situ vaccine production can be achieved without requiring systemic administration of vaccine formulations. The skin provides an accessible tissue target for electrode placement, and the constant-power approach compensates for variations in skin electrical conductivity that can occur between patients and between different anatomical locations on the same patient.

The biological tissue can comprise bone marrow tissue for gene electrotransfer. Bone marrow contains hematopoietic stem cells and progenitor cells that give rise to blood cells and immune cells. Gene electrotransfer to bone marrow tissue can deliver genetic material to hematopoietic stem cells for treating hematological disorders and genetic diseases affecting blood cell lineages. The constant-power electroporation techniques can achieve consistent gene delivery to bone marrow cells despite variations in bone marrow electrical properties that can occur between patients and between different bone marrow compartments.

The biological tissue can comprise liver tissue for gene electrotransfer to express stem cell genes or genes associated with regeneration. Liver tissue has regenerative capacity, and gene electrotransfer can enhance liver regeneration by delivering genetic material encoding growth factors, transcription factors, or other proteins that promote hepatocyte proliferation and tissue repair. The constant-power electroporation techniques can deliver genetic material to hepatocytes and other liver cell types to express stem cell genes or genes associated with regeneration. Liver tissue electrical properties can vary based on disease state, including differences between healthy liver tissue, fibrotic liver tissue, and cirrhotic liver tissue. The constant-power approach compensates for these variations in liver tissue electrical conductivity to achieve consistent gene delivery outcomes.

The biological tissue can comprise cardiac tissue for gene electrotransfer to express stem cell genes or genes associated with regeneration. Cardiac tissue has limited regenerative capacity following injury, and gene electrotransfer can deliver genetic material encoding factors that promote cardiomyocyte proliferation, survival, or regeneration. The constant-power electroporation techniques can deliver genetic material to cardiac tissue to express stem cell genes or genes associated with regeneration. Cardiac tissue electrical properties can vary between patients and between different regions of the heart, including differences between healthy myocardium, ischemic tissue, and scar tissue. The constant-power approach compensates for these variations to achieve consistent gene delivery to cardiac tissue.

The biological tissue can comprise liver tissue or cardiac tissue for downregulating expression of fibrosis or senescent genes. Fibrosis in liver tissue and cardiac tissue can impair organ function and contribute to disease progression. Gene electrotransfer can deliver genetic material encoding siRNA, shRNA, or CRISPR components that downregulate expression of genes involved in fibrotic pathways or cellular senescence. The constant-power electroporation techniques can achieve consistent delivery of gene silencing constructs to liver tissue or cardiac tissue to reduce fibrosis or senescent cell accumulation. By downregulating expression of fibrosis genes, tissue remodeling can be reduced, and organ function can be preserved or improved. By downregulating expression of senescent genes, accumulation of senescent cells that contribute to tissue dysfunction can be reduced.

The biological tissue can comprise neural tissue for direct gene delivery for conditions including Parkinson's disease, ALS, and Huntington's disease. Neural tissue presents challenges for gene delivery due to the blood-brain barrier and the sensitivity of neural cells to damage. The constant-power electroporation techniques can enable localized gene therapy for neural tissue by delivering genetic material encoding therapeutic proteins, neurotrophic factors, or gene editing components directly to affected brain regions or spinal cord tissue. For Parkinson's disease, gene electrotransfer can deliver genetic material encoding dopamine-synthesizing enzymes or neurotrophic factors to substantia nigra or striatal tissue. For ALS (amyotrophic lateral sclerosis), gene electrotransfer can deliver genetic material encoding neuroprotective factors or gene silencing constructs targeting disease-associated genes to motor neurons or surrounding glial cells. For Huntington's disease, gene electrotransfer can deliver genetic material encoding gene silencing constructs targeting the mutant huntingtin gene. The constant-power approach compensates for variations in neural tissue electrical properties to achieve consistent gene delivery while minimizing damage to sensitive neural structures.

The biological tissue can comprise spleen tissue for T-cell transfection. The spleen contains T-cells and other immune cells and serves as a secondary lymphoid organ where immune responses are initiated and regulated. Gene electrotransfer to spleen tissue can deliver genetic material to T-cells residing within the spleen, providing an alternative or complementary approach to lymph node gene electrotransfer for in situ T-cell engineering. The constant-power electroporation techniques can achieve consistent T-cell transfection within spleen tissue despite variations in spleen electrical properties. The spleen can be accessed surgically or through minimally invasive approaches for electrode placement and gene electrotransfer.

The biological tissue can comprise cancerous tissue for knocking genes into tumor cells for immune recognition. Tumor cells can evade immune recognition through various mechanisms including downregulation of major histocompatibility complex molecules and expression of immune checkpoint ligands. Gene electrotransfer to cancerous tissue can deliver genetic material encoding immunostimulatory molecules, tumor antigens, or other factors that enhance immune recognition of tumor cells. The constant-power electroporation techniques can deliver genetic material to tumor cells to knock in genes that promote immune recognition and tumor cell killing by the immune system. Tumor tissue electrical properties can vary based on tumor type, tumor stage, and the presence of necrotic or hypoxic regions within the tumor. The constant-power approach compensates for these variations to achieve consistent gene delivery throughout the tumor mass.

The biological tissue can comprise injured tissue for delivering growth factors or reprogramming factors to stimulate regeneration including cardiac tissue post-myocardial infarction or musculoskeletal injuries. Following myocardial infarction, cardiac tissue undergoes remodeling that can lead to scar formation and impaired cardiac function. Gene electrotransfer to cardiac tissue post-myocardial infarction can deliver genetic material encoding growth factors, angiogenic factors, or reprogramming factors that promote cardiomyocyte survival, proliferation, or regeneration. For musculoskeletal injuries including muscle tears, tendon injuries, and ligament injuries, gene electrotransfer can deliver genetic material encoding growth factors or regenerative factors that promote tissue repair and functional recovery. The constant-power electroporation techniques can achieve consistent gene delivery to injured tissue despite variations in tissue electrical properties that can occur in injured and healing tissue compared to healthy tissue.

The biological tissue can comprise wound tissue or skin graft tissue for delivering pro-regenerative genes to improve healing and integration. Wound healing involves complex processes including inflammation, proliferation, and remodeling. Gene electrotransfer to wound tissue can deliver genetic material encoding growth factors, cytokines, or other factors that promote wound healing and reduce scar formation. For skin graft applications, gene electrotransfer can deliver genetic material to the graft tissue or the recipient wound bed to improve graft integration and survival. The constant-power electroporation techniques can achieve consistent gene delivery to wound tissue or skin graft tissue to improve healing outcomes and graft integration.

The biological tissue can comprise cells targeted for in situ reprogramming to produce induced pluripotent stem cells or other therapeutic cell types without ex vivo manipulation. Cellular reprogramming involves delivering transcription factors or other factors that convert differentiated cells into pluripotent stem cells or other cell types. Gene electrotransfer can deliver genetic material encoding reprogramming factors including Oct4, Sox2, Klf4, and c-Myc to target cells within tissue to achieve in situ reprogramming. The constant-power electroporation techniques can deliver reprogramming factors to cells within tissue to produce induced pluripotent stem cells or other therapeutic cell types directly within the patient without requiring cell isolation, ex vivo culture, and reinfusion. In situ reprogramming can generate therapeutic cell types at the site where the cells are needed, avoiding the complexity and cost associated with ex vivo cell manipulation and the challenges of cell delivery and engraftment following reinfusion.

The biological tissue can comprise embryos for transfection using constant power density or constant current density for generating genetically modified organisms. Embryo transfection involves delivering genetic material to cells within developing embryos to introduce heritable genetic modifications. The constant-power electroporation techniques described herein can deliver genetic material to embryonic cells with consistent transfection outcomes regardless of variations in embryo electrical properties. Embryo electrical properties can vary based on species, developmental stage, and individual embryo characteristics. The constant-power approach compensates for these variations to achieve consistent gene delivery across multiple embryos within a transfection batch. For generating genetically modified organisms, genetic material encoding desired traits, reporter genes, or gene editing components can be delivered to embryos via constant-power electroporation. The embryos can then develop into organisms carrying the introduced genetic modifications. Constant current density can also be used for embryo transfection, where the current delivered to the embryo is maintained at a substantially constant level during pulse delivery. The constant current density approach provides consistent current distribution within the embryo tissue regardless of variations in embryo electrical conductivity. Embryo transfection using constant power density or constant current density can be applied to embryos from various species including mammalian embryos, avian embryos, fish embryos, and invertebrate embryos for research applications and for generating genetically modified organisms for agricultural, biomedical, and industrial purposes.

The biological tissue can comprise livestock tissue for enhancing traits such as disease resistance or productivity via targeted gene delivery. Livestock animals including cattle, pigs, sheep, goats, poultry, and fish can benefit from genetic modifications that enhance economically valuable traits. Disease resistance traits can reduce livestock mortality, decrease antibiotic use, and improve animal welfare. Productivity traits can include increased growth rate, improved feed conversion efficiency, enhanced milk production, improved meat quality, and increased reproductive performance. The constant-power electroporation techniques described herein can deliver genetic material to livestock tissue to introduce genetic modifications that enhance disease resistance or productivity. Gene delivery to livestock tissue can target somatic cells for transient or localized effects, or can target germline cells or embryos for heritable modifications. The constant-power approach compensates for variations in livestock tissue electrical properties that can occur between individual animals, between different tissue types within the same animal, and between different breeds or species. Targeted gene delivery to livestock tissue can deliver genetic material encoding disease resistance genes, growth factors, metabolic enzymes, or other proteins that enhance desired traits. Gene editing components including CRISPR/Cas9 ribonucleoproteins can be delivered to livestock tissue via constant-power electroporation to introduce precise genetic modifications at specific genomic loci. The constant-power electroporation techniques provide scalability for livestock applications where large numbers of animals or embryos can require treatment with consistent outcomes across the treated population.

The biological tissue can comprise plant tissue for delivering DNA with greater consistency and scalability for developing pest-resistant and climate-adapted crops. Plant genetic engineering involves delivering genetic material to plant cells to introduce traits that improve crop performance, nutritional value, or environmental adaptation. Pest-resistant crops can reduce pesticide use, decrease crop losses, and improve agricultural sustainability. Climate-adapted crops can tolerate drought, heat, cold, salinity, or other environmental stresses that limit crop productivity in changing climate conditions. The constant-power electroporation techniques described herein can deliver DNA to plant tissue with greater consistency and scalability compared to conventional plant transformation methods. Plant tissue electrical properties can vary based on plant species, tissue type, developmental stage, and physiological state. The constant-power approach compensates for these variations to achieve consistent DNA delivery across different plant tissues and across multiple samples within a transformation experiment. DNA delivery to plant tissue can target protoplasts, callus tissue, meristematic tissue, or other plant cell types suitable for regeneration of transformed plants. The constant-power electroporation techniques can deliver plasmid DNA encoding pest resistance genes, stress tolerance genes, or other traits to plant cells for stable integration into the plant genome. Gene editing components including CRISPR/Cas9 ribonucleoproteins can be delivered to plant tissue via constant-power electroporation to introduce precise genetic modifications for crop improvement. The scalability of constant-power electroporation enables high-throughput plant transformation for developing pest-resistant and climate-adapted crop varieties. The consistency of constant-power electroporation reduces variability in transformation efficiency, enabling more predictable outcomes in plant genetic engineering programs.

The disclosed technology can be further understood according to the following clauses:

Clause 1: A method for inducing electroporation in biological tissue, comprising: positioning at least one electrode in proximity to the biological tissue; delivering a pulsed electric field to the biological tissue through the at least one electrode; monitoring a voltage and a current delivered to the biological tissue during delivery of the pulsed electric field; and adjusting at least one of the voltage or the current to maintain a substantially constant power delivered to the biological tissue, wherein the substantially constant power produces a substantially constant power density distribution within the biological tissue that is independent of electrical conductivity variations in the biological tissue.

Clause 2: The method of clause 1, wherein the substantially constant power density distribution produces substantially consistent reversible electroporation areas and substantially consistent irreversible electroporation areas within the biological tissue regardless of the electrical conductivity variations.

Clause 3: The method of clause 2, wherein the substantially constant power density is in a range from 1,000 W/cm³ to 10,000 W/cm³.

Clause 4: The method of clause 1, further comprising: delivering a preliminary low-voltage pulse to the biological tissue prior to delivering the pulsed electric field; measuring a response to the preliminary low-voltage pulse to determine a bulk electrical conductivity of the biological tissue; and calculating an initial voltage or current setting based on the determined bulk electrical conductivity to achieve a target power.

Clause 5: The method of clause 1, wherein adjusting at least one of the voltage or the current comprises: calculating an instantaneous power as a product of the monitored voltage and the monitored current; and modifying the current to maintain the instantaneous power at a target power value.

Clause 6: The method of clause 5, wherein modifying the current comprises operating a transistor in a linear mode to regulate current flow through the biological tissue.

Clause 7: The method of clause 1, wherein the pulsed electric field comprises monophasic pulses, biphasic pulses, or a combination thereof.

Clause 8: The method of clause 1, wherein the pulsed electric field comprises pulses having a pulse duration in a range from 500 nanoseconds to 100 microseconds.

Clause 9: The method of clause 1, wherein the at least one electrode comprises a plurality of needle electrodes arranged in an array configuration.

Clause 10: The method of clause 1, further comprising monitoring a bioimpedance of the biological tissue during delivery of the pulsed electric field to determine an electroporation status of the biological tissue.

Clause 11: The method of clause 10, further comprising terminating delivery of the pulsed electric field based on the electroporation status indicating a predetermined level of electroporation has been achieved.

Clause 12: A system for delivering pulsed electric fields to biological tissue, comprising: at least one electrode configured to be positioned in proximity to the biological tissue; a pulse generator electrically coupled to the at least one electrode and configured to deliver a pulsed electric field to the biological tissue; a sensing circuit configured to measure a voltage and a current delivered to the biological tissue; and a controller coupled to the sensing circuit and the pulse generator, the controller configured to adjust at least one of the voltage or the current delivered by the pulse generator to maintain a substantially constant power delivered to the biological tissue, wherein the substantially constant power produces a substantially constant power density distribution within the biological tissue that is independent of electrical conductivity variations in the biological tissue.

Clause 13: The system of clause 12, wherein the pulse generator comprises a full-bridge topology including a plurality of transistors arranged in an H-bridge configuration, wherein at least one transistor of the plurality of transistors is configured to operate in a linear mode to regulate current flow through the biological tissue.

Clause 14: The system of clause 13, wherein the sensing circuit comprises a sense resistor positioned in a common low-side return path of the full-bridge topology, wherein load current flows through the sense resistor in a same direction for both positive and negative pulse polarities.

Clause 15: The system of clause 12, wherein the controller is configured to: calculate an instantaneous power as a product of the measured voltage and the measured current; and adjust the current delivered by the pulse generator to maintain the instantaneous power at a target power value by dividing the target power value by the measured voltage to determine a reference current setpoint.

Clause 16: The system of clause 12, wherein the controller comprises an analog divider configured to calculate a reference current setpoint in real-time, or a digital processor configured to calculate the reference current setpoint using a digital-to-analog converter.

Clause 17: A method for in situ transfection of T-cells within a lymph node, comprising: injecting a genetic material into the lymph node, wherein the genetic material comprises at least one of DNA, mRNA, or a CRISPR component; positioning at least one electrode in proximity to the lymph node; delivering a pulsed electric field to the lymph node through the at least one electrode to induce reversible electroporation of T-cells within the lymph node; and controlling a power delivered to the lymph node during delivery of the pulsed electric field to maintain a substantially constant power density within the lymph node, thereby facilitating uptake of the genetic material by the T-cells.

Clause 18: The method of clause 17, wherein the genetic material comprises mRNA encoding a chimeric antigen receptor, and wherein uptake of the mRNA by the T-cells produces autologous CAR T-cells within the lymph node.

Clause 19: The method of clause 18, wherein the mRNA further encodes a suicide gene under a T-cell specific promoter, wherein the T-cell specific promoter comprises a dLck promoter or a CD3δ promoter.

Clause 20: The method of clause 17, wherein controlling the power delivered to the lymph node comprises: monitoring a voltage and a current delivered to the lymph node during delivery of the pulsed electric field; calculating an instantaneous power as a product of the monitored voltage and the monitored current; and adjusting at least one of the voltage or the current to maintain the instantaneous power at a target power value corresponding to a power density in a range from 1,000 W/cm³ to 10,000 W/cm³.

A number of implementations have been described. Nevertheless, it will be understood that various modifications may be made without departing from the spirit and scope of the disclosure. Accordingly, other implementations are within the scope of the following claims.

1. A method for inducing electroporation in biological tissue, comprising:
positioning at least one electrode in proximity to the biological tissue;
delivering a pulsed electric field to the biological tissue through the at least one electrode;
monitoring a voltage and a current delivered to the biological tissue during delivery of the pulsed electric field; and
adjusting at least one of the voltage or the current to maintain a substantially constant power delivered to the biological tissue, wherein the substantially constant power produces a substantially constant power density distribution within the biological tissue that is independent of electrical conductivity variations in the biological tissue.

2. The method of claim 1, wherein the substantially constant power density distribution produces substantially consistent reversible electroporation areas and substantially consistent irreversible electroporation areas within the biological tissue regardless of the electrical conductivity variations.

3. The method of claim 2, wherein the substantially constant power density is in a range from 1,000 W/cm³ to 10,000 W/cm³.

4. The method of claim 1, further comprising:
delivering a preliminary low-voltage pulse to the biological tissue prior to delivering the pulsed electric field;
measuring a response to the preliminary low-voltage pulse to determine a bulk electrical conductivity of the biological tissue; and
calculating an initial voltage or current setting based on the determined bulk electrical conductivity to achieve a target power.

5. The method of claim 1, wherein adjusting at least one of the voltage or the current comprises:
calculating an instantaneous power as a product of the monitored voltage and the monitored current; and
modifying the current to maintain the instantaneous power at a target power value.

6. The method of claim 5, wherein modifying the current comprises operating a transistor in a linear mode to regulate current flow through the biological tissue.

7. The method of claim 1, wherein the pulsed electric field comprises monophasic pulses, biphasic pulses, or a combination thereof.

8. The method of claim 1, wherein the pulsed electric field comprises pulses having a pulse duration in a range from 500 nanoseconds to 100 microseconds.

9. The method of claim 1, wherein the at least one electrode comprises a plurality of needle electrodes arranged in an array configuration.

10. The method of claim 1, further comprising monitoring a bioimpedance of the biological tissue during delivery of the pulsed electric field to determine an electroporation status of the biological tissue.

11. The method of claim 10, further comprising terminating delivery of the pulsed electric field based on the electroporation status indicating a predetermined level of electroporation has been achieved.

12. A system for delivering pulsed electric fields to biological tissue, comprising:
at least one electrode configured to be positioned in proximity to the biological tissue;
a pulse generator electrically coupled to the at least one electrode and configured to deliver a pulsed electric field to the biological tissue;
a sensing circuit configured to measure a voltage and a current delivered to the biological tissue; and
a controller coupled to the sensing circuit and the pulse generator, the controller configured to adjust at least one of the voltage or the current delivered by the pulse generator to maintain a substantially constant power delivered to the biological tissue, wherein the substantially constant power produces a substantially constant power density distribution within the biological tissue that is independent of electrical conductivity variations in the biological tissue.

13. The system of claim 12, wherein the pulse generator comprises a full-bridge topology including a plurality of transistors arranged in an H-bridge configuration, wherein at least one transistor of the plurality of transistors is configured to operate in a linear mode to regulate current flow through the biological tissue.

14. The system of claim 13, wherein the sensing circuit comprises a sense resistor positioned in a common low-side return path of the full-bridge topology, wherein load current flows through the sense resistor in a same direction for both positive and negative pulse polarities.

15. The system of claim 12, wherein the controller is configured to:
calculate an instantaneous power as a product of the measured voltage and the measured current; and
adjust the current delivered by the pulse generator to maintain the instantaneous power at a target power value by dividing the target power value by the measured voltage to determine a reference current setpoint.

16. The system of claim 12, wherein the controller comprises an analog divider configured to calculate a reference current setpoint in real-time, or a digital processor configured to calculate the reference current setpoint using a digital-to-analog converter.

17. A method for in situ transfection of T-cells within a lymph node, comprising:
injecting a genetic material into the lymph node, wherein the genetic material comprises at least one of DNA, mRNA, or a CRISPR component;
positioning at least one electrode in proximity to the lymph node;
delivering a pulsed electric field to the lymph node through the at least one electrode to induce reversible electroporation of T-cells within the lymph node; and
controlling a power delivered to the lymph node during delivery of the pulsed electric field to maintain a substantially constant power density within the lymph node, thereby facilitating uptake of the genetic material by the T-cells.

18. The method of claim 17, wherein the genetic material comprises mRNA encoding a chimeric antigen receptor, and wherein uptake of the mRNA by the T-cells produces autologous CAR T-cells within the lymph node.

19. The method of claim 18, wherein the mRNA further encodes a suicide gene under a T-cell specific promoter, wherein the T-cell specific promoter comprises a dLck promoter or a CD3δ promoter.

20. The method of claim 17, wherein controlling the power delivered to the lymph node comprises:
monitoring a voltage and a current delivered to the lymph node during delivery of the pulsed electric field;
calculating an instantaneous power as a product of the monitored voltage and the monitored current; and
adjusting at least one of the voltage or the current to maintain the instantaneous power at a target power value corresponding to a power density in a range from 1,000 W/cm³ to 10,000 W/cm³.
